SEQUENCE LISTING (34 sequences)

SEQ ID NO: 1 - protein, 107 aa, synthetic construct [sequence omitted]

SEQ ID NO: 2 - DNA, 321 bp, synthetic construct
  source 1..321: mol_type = other DNA
  misc_feature 1..321: note = Initial 4D5 VL nucleotide
gatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggatccagatctgggacggatttcactctgaccatcagcagtctgcagccggaagacttcgcaacttattactgtcagcaacattatactactcctcccacgttcggacagggtaccaaggtggagatcaaa

SEQ ID NO: 3 - protein, 107 aa, synthetic construct
  REGION 1..107: note = VL-T91N AA
DIQMTQSPSSLSASVGDRVTITCRASQDVNTAVAWYQQKPGKAPKLLIYSASFLYSGVPSRFSGSRSGTDFNLTISSLQPEDFATYYCQQHYNTPPTFGQGTKVEIK

SEQ ID NO: 4 - DNA, 321 bp, synthetic construct
  source 1..321: mol_type = other DNA
  misc_feature 1..321: note = VL-T91N nucleotide
gatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggatccagatctgggacggatttcaatctgaccatcagcagtctgcagccggaagacttcgcaacttattactgtcagcaacattatactactcctcccacgttcggacagggtaccaaggtggagatcaaa

SEQ ID NO: 5 - protein, 107 aa, synthetic construct
  REGION 1..107: note = VL-T93 AA
DIQMTQSPSSLSASVGDRVTITCRASQDVNTAVAWYQQKPGKAPKLLIYSASFLYSGVPSRFSGSRSGTDFTLNISSLQPEDFATYYCQQHYTTPPTFGQGTKVEIK

SEQ ID NO: 6 - DNA, 321 bp, synthetic construct
  source 1..321: mol_type = other DNA
  misc_feature 1..321: note = VL-T93 nucleotide
gatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggatccagatctgggacggatttcactctgaacatcagcagtctgcagccggaagacttcgcaacttattactgtcagcaacattatactactcctcccacgttcggacagggtaccaaggtggagatcaaa

SEQ ID NO: 7 - protein, 107 aa, synthetic construct
  REGION 1..107: note = VL--T91N, T93N AA
DIQMTQSPSSLSASVGDRVTITCRASQDVNTAVAWYQQKPGKAPKLLIYSASFLYSGVPSRFSGSRSGTDFNLNISSLQPEDFATYYCQQHYNTPPTFGQGTKVEIK

SEQ ID NO: 8 - DNA, 321 bp, synthetic construct
  source 1..321: mol_type = other DNA
  misc_feature 1..321: note = VL--T91N, T93N nucleotide
gatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggatccagatctgggacggatttcaatctgaacatcagcagtctgcagccggaagacttcgcaacttattactgtcagcaacattatactactcctcccacgttcggacagggtaccaaggtggagatcaaa

SEQ ID NO: 9 - protein, 123 aa, synthetic construct
  REGION 1..123: note = Initial 4D5-VH AA
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSRAA

SEQ ID NO: 10 - DNA, 369 bp, synthetic construct
  source 1..369: mol_type = other DNA
  misc_feature 1..369: note = Initial 4D5-VH nucleotide
gaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcctcgcgtgcggcc

SEQ ID NO: 11 - protein, 245 aa, synthetic construct
  REGION 1..245: note = Initial 4D5-VL,VH AA
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSRAA

SEQ ID NO: 12 - DNA, 735 bp, synthetic construct
  source 1..735: mol_type = other DNA
  misc_feature 1..735: note = Initial 4D5-VL,VH nucleotide
gatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggatccagatctgggacggatttcactctgaccatcagcagtctgcagccggaagacttcgcaacttattactgtcagcaacattatactactcctcccacgttcggacagggtaccaaggtggagatcaaaggtggaggtggatcaggtggaggtggatcaggtggaggtggatcagaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcctcgcgtgcggcc

SEQ ID NO: 13 - protein, 287 aa, synthetic construct
  REGION 1..287: note = 4D5-VL,VH with T91N AA, with linkers
SSGGGGSGGGGSGGGGSASDIQMTQSPSSLSASVGDRVTITCRASQDVNTAVAWYQQKPGKAPKLLIYSASFLYSGVPSRFSGSRSGTDFNLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYLTNGYTRYADSVKGRFTISADTSKNTTYLQMNSLRAEDTAVYYCSRWGGDGFYAMDYWGQGTLVTVSSRAAIDAKTTAPSVYPLALVSSGSGSL

SEQ ID NO: 14 - DNA, 861 bp, synthetic construct
  source 1..861: mol_type = other DNA
  misc_feature 1..861: note = 4D5-VL,VH with T91N nucleotide with linkers
agctcaggtggaggcggttcaggcggaggtggctctggcggtggcggatctgctagcgatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggatccagatctgggacggatttcaatctgaccatcagcagtctgcagccggaagacttcgcaacttattactgtcagcaacattatactactcctcccacgttcggacagggtaccaaggtggagatcaaaggtggaggtggatcaggtggaggtggatcaggtggaggtggatcagaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcttacgaatggttatactagatatgccgatagcgtcaagggccgtttcactataagcgcagacacatccaaaaacacaacctacctgcagatgaacagtctgcgtgctgaggacactgccgtctattattgttctagatggggaggggacggcttctatgctatggactactggggtcaaggaaccctggtcacagtctcctcgcgtgcggccatcgatgctaaaacgacggcaccgtctgtctacccactggcacttgtctcgtctggatccgggtctctg

SEQ ID NO: 15 - protein, 287 aa, synthetic construct
  REGION 1..287: note = 4D5-VL,VH with T93N AA with linkers
SSGGGGSGGGGSGGGGSASDIQMTQSPSSLSASVGDRVTITCRASQDVNTAVAWYQQKPGKAPKLLIYSASFLYSGVPSRFSGSRSGTDFTLNISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYLTNGYTRYADSVKGRFTISADTSKNTTYLQMNSLRAEDTAVYYCSRWGGDGFYAMDYWGQGTLVTVSSRAAIDAKTTAPSVYPLALVSSGSGSL

SEQ ID NO: 16 - DNA, 861 bp, synthetic construct
  source 1..861: mol_type = other DNA
  misc_feature 1..861: note = 4D5-VL,VH with T93N nucleotide with linkers
agctcaggtggaggcggttcaggcggaggtggctctggcggtggcggatctgctagcgatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggatccagatctgggacggatttcactctgaacatcagcagtctgcagccggaagacttcgcaacttattactgtcagcaacattatactactcctcccacgttcggacagggtaccaaggtggagatcaaaggtggaggtggatcaggtggaggtggatcaggtggaggtggatcagaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcttacgaatggttatactagatatgccgatagcgtcaagggccgtttcactataagcgcagacacatccaaaaacacaacctacctgcagatgaacagtctgcgtgctgaggacactgccgtctattattgttctagatggggaggggacggcttctatgctatggactactggggtcaaggaaccctggtcacagtctcctcgcgtgcggccatcgatgctaaaacgacggcaccgtctgtctacccactggcacttgtctcgtctggatccgggtctctg

SEQ ID NO: 17 - protein, 287 aa, synthetic construct
  REGION 1..287: note = 4D5-VL,VH with T91N, T93N AA with linkers
SSGGGGSGGGGSGGGGSASDIQMTQSPSSLSASVGDRVTITCRASQDVNTAVAWYQQKPGKAPKLLIYSASFLYSGVPSRFSGSRSGTDFNLNISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYLTNGYTRYADSVKGRFTISADTSKNTTYLQMNSLRAEDTAVYYCSRWGGDGFYAMDYWGQGTLVTVSSRAAIDAKTTAPSVYPLALVSSGSGSL

SEQ ID NO: 18 - DNA, 861 bp, synthetic construct
  source 1..861: mol_type = other DNA
  misc_feature 1..861: note = 4D5-VL,VH with T91, T93N nucleotide with linkers
agctcaggtggaggcggttcaggcggaggtggctctggcggtggcggatctgctagcgatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggatccagatctgggacggatttcaatctgaacatcagcagtctgcagccggaagacttcgcaacttattactgtcagcaacattatactactcctcccacgttcggacagggtaccaaggtggagatcaaaggtggaggtggatcaggtggaggtggatcaggtggaggtggatcagaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcttacgaatggttatactagatatgccgatagcgtcaagggccgtttcactataagcgcagacacatccaaaaacacaacctacctgcagatgaacagtctgcgtgctgaggacactgccgtctattattgttctagatggggaggggacggcttctatgctatggactactggggtcaaggaaccctggtcacagtctcctcgcgtgcggccatcgatgctaaaacgacggcaccgtctgtctacccactggcacttgtctcgtctggatccgggtctctg

SEQ ID NO: 19 - protein, 511 aa, synthetic construct
  REGION 1..511: note = VSV G with signal sequence AA
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 20 - DNA, 1536 bp, synthetic construct
  source 1..1536: mol_type = other DNA
  misc_feature 1..1536: note = VSV G with signal sequence nucleotide
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 21 - protein, 495 aa, synthetic construct
  REGION 1..495: note = VSV G without signal sequence AA
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 22 - DNA, 1488 bp, synthetic construct
  source 1..1488: mol_type = other DNA
  misc_feature 1..1488: note = VSV G without signal sequence nucleotide
aagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 23 - protein, 511 aa, synthetic construct
  REGION 1..511: note = VSV G with 2 constructed mutations (K47 and R354) AA
MKCLLYLAFLFIGVNCKFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 24 - DNA, 1536 bp, synthetic construct
  source 1..1536: mol_type = other DNA
  misc_feature 1..1536: note = VSV G with 2 constructed mutations (K47 and R354) nucleotide
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgccccagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaacaggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 25 - protein, 495 aa, synthetic construct
  REGION 1..495: note = VSV G with constructed and adapted mutations (Q10K, K47Q, Y156D, M184T, E194K, E238K, R354Q, H397R) AA
KFTIVFPHNKKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNDICPTVHNSTTWHSDYKVKGLCDSNLISTDITFFSEDGKLSSLGKEGTGFRSNYFAYETGGKACKMQYCKHWGVRLPSGVWFKMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLRLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 26 - DNA, 1488 bp, synthetic construct
  source 1..1488: mol_type = other DNA
  misc_feature 1..1488: note = VSV G with constructed and adapted mutations (Q10K, K47Q, Y156D, M184T, E194K, E238K, R354Q, H397R) nucleotide
aagttcaccatagtttttccacacaacaagaaaggaaactggaaaaatgttccttctaattaccattattgcccgtcaagctcagatttaaattggcataatgacttaataggcacagccttacaagtcaaaatgccccagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaatgacatatgccccactgtccataactctacaacctggcattctgactataaggtcaaagggctatgtgattctaacctcatttccacggacatcaccttcttctcagaggacggaaagctatcatccctgggaaaggagggcacagggttcagaagtaactactttgcttatgaaactggaggcaaggcctgcaaaatgcaatactgcaagcattggggagtcagactcccatcaggtgtctggttcaagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaacaggaactgtgggatgactgggcaccatatgaagacgtggaaattggacccaatggagttctgaggaccagttcaggatataagtttcctttatacatgattggacatggtatgttggactccgatcttcgtcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 27 - protein, 798 aa, synthetic construct
  REGION 1..798: note = Compound SCA-G protein with all constructed and adapted mutations including start site (T91N, T93N, P278L, Q10K, K47Q, Y156D, M184T, E194K, E238K, R354Q, H397R) AA
MKCLLYLAFLFIGVNCSSGGGGSGGGGSGGGGSASDIQMTQSPSSLSASVGDRVTITCRASQDVNTAVAWYQQKPGKAPKLLIYSASFLYSGVPSRFSGSRSGTDFNLNISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYLTNGYTRYADSVKGRFTISADTSKNTTYLQMNSLRAEDTAVYYCSRWGGDGFYAMDYWGQGTLVTVSSRAAIDAKTTAPSVYPLALVSSGSGSLKFTIVFPHNKKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNDICPTVHNSTTWHSDYKVKGLCDSNLISTDITFFSEDGKLSSLGKEGTGFRSNYFAYETGGKACKMQYCKHWGVRLPSGVWFKMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLRLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 28 - DNA, 2397 bp, synthetic construct
  source 1..2397: mol_type = other DNA
  misc_feature 1..2397: note = Compound SCA-G protein with all constructed and adapted mutations including start site (T91N, T93N, P278L, Q10K, K47Q, Y156D, M184T, E194K, E238K, R354Q, H397R) nucleotide
atgaagtgccttttgtacttagcctttttattcattggggtgaattgcagctcaggtggaggcggttcaggcggaggtggctctggcggtggcggatctgctagcgatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggatccagatctgggacggatttcaatctgaacatcagcagtctgcagccggaagacttcgcaacttattactgtcagcaacattatactactcctcccacgttcggacagggtaccaaggtggagatcaaaggtggaggtggatcaggtggaggtggatcaggtggaggtggatcagaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcttacgaatggttatactagatatgccgatagcgtcaagggccgtttcactataagcgcagacacatccaaaaacacaacctacctgcagatgaacagtctgcgtgctgaggacactgccgtctattattgttctagatggggaggggacggcttctatgctatggactactggggtcaaggaaccctggtcacagtctcctcgcgtgcggccatcgatgctaaaacgacggcaccgtctgtctacccactggcacttgtctcgtctggatccgggtctctgaagttcaccatagtttttccacacaacaagaaaggaaactggaaaaatgttccttctaattaccattattgcccgtcaagctcagatttaaattggcataatgacttaataggcacagccttacaagtcaaaatgccccagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaatgacatatgccccactgtccataactctacaacctggcattctgactataaggtcaaagggctatgtgattctaacctcatttccacggacatcaccttcttctcagaggacggaaagctatcatccctgggaaaggagggcacagggttcagaagtaactactttgcttatgaaactggaggcaaggcctgcaaaatgcaatactgcaagcattggggagtcagactcccatcaggtgtctggttcaagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaacaggaactgtgggatgactgggcaccatatgaagacgtggaaattggacccaatggagttctgaggaccagttcaggatataagtttcctttatacatgattggacatggtatgttggactccgatcttcgtcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 29 - protein, 19 aa, synthetic construct
  REGION 1..19: note = Linker 1 preceding SCA AA
SSGGGGSGGGGSGGGGSAS

SEQ ID NO: 30 - DNA, 57 bp, synthetic construct
  source 1..57: mol_type = other DNA
  misc_feature 1..57: note = Linker 1 preceding SCA nucleotide
agctcaggtggaggcggttcaggcggaggtggctctggcggtggcggatctgctagc

SEQ ID NO: 31 - protein, 23 aa, synthetic construct
  REGION 1..23: note = Linker 2 following SCA AA
IDAKTTAPSVYPLAPVSSGSGSL

SEQ ID NO: 32 - DNA, 69 bp, synthetic construct
  source 1..69: mol_type = other DNA
  misc_feature 1..69: note = Linker 2 following SCA nucleotide
atcgatgctaaaacgacggcaccgtctgtctacccactggcacctgtctcgtctggatccgggtctctg

SEQ ID NO: 33 - protein, 23 aa, synthetic construct
  REGION 1..23: note = Linker 2 mutated following SCA AA
IDAKTTAPSVYPLALVSSGSGSL

SEQ ID NO: 34 - DNA, 69 bp, synthetic construct
  source 1..69: mol_type = other DNA
  misc_feature 1..69: note = Linker 2 mutated following SCA nucleotide
atcgatgctaaaacgacggcaccgtctgtctacccactggcacttgtctcgtctggatccgggtctctg

(19) United States
(12) Patent Application Publication
(10) Pub. No.: US 2026/0103727 A1
(43) Pub. Date: Apr. 16, 2026

(54) THERAPEUTIC RECOMBINANT VIRUSES

(71) Applicant: University of Pittsburgh - Of The Commonwealth System of Higher Education, Pittsburgh, PA (US)

(72) Inventors: Ira Bergman, Pittsburgh, PA (US); Yanhua Gao, Wexford, PA (US)

(73) Assignee: University of Pittsburgh - Of The Commonwealth System of Higher Education, Pittsburgh, PA (US)

(21) Appl. No.: 19/115,107

(22) PCT Filed: Sep. 26, 2023
(86) PCT No.: PCT/US2023/075116

§ 371 Date: Mar. 25, 2025

Related U.S. Application Data
(60) Provisional application No. 63/410,329, filed on Sep. 27, 2022.

(51) Int. Cl. C12N 15/86; A61P 35/00; C07K 14/005; C07K 16/30; C07K 16/32; C12N 7/00

(52) U.S. Cl. CPC C12N 15/86; A61P 35/00; C07K 14/005; C07K 16/3069; C07K 16/32; C12N 7/00; C12N 2760/20222; C12N 2760/20243; C12N 2810/859

(57) ABSTRACT

The present disclosure provides recombinant viruses comprising recombinant target surface molecules. The present disclosure further provides pharmaceutical compositions comprising the recombinant viruses and methods of using the viruses and pharmaceutical compositions for treating or preventing conditions or disorders.

### CROSS-REFERENCE TO PRIOR APPLICATION

This application claims benefit to U.S. Provisional Patent Application No. 63/410,329, filed Sep. 27, 2022, which is hereby incorporated by reference in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 62,284 Byte Extensible Markup Language (XML) file named “767712_ST26.xml,” created on Sep. 22, 2023.

### BACKGROUND

Some viruses have been useful for therapeutic purposes (e.g., cancer therapy), however, safe and effective treatments are still needed.

### SUMMARY OF THE INVENTION

The present disclosure provides a recombinant virus (e.g., a Vesicular Stomatitis Virus (VSV)) comprising a recombinant target surface molecule (e.g., a recombinant target surface molecule or protein that targets HER2 or PSMA). In certain aspects of the invention, the recombinant target surface molecule comprises an antibody or antigen-binding fragment thereof.

In certain aspects of the invention, the antibody or antigen-binding fragment thereof comprises a variable light chain comprising an amino acid sequence that is at least about 80% homologous or identical to the amino acid sequence set forth in SEQ ID NO: 1, SEQ ID NO: 3, SEQ ID NO: 5, or SEQ ID NO: 7. In certain aspects of the invention, the antibody or antigen-binding fragment thereof comprises a variable light chain comprising the amino acid sequence set forth in SEQ ID NO: 1, SEQ ID NO: 3, SEQ ID NO: 5, or SEQ ID NO: 7. In certain aspects of the invention, the antibody or antigen-binding fragment thereof comprises a variable light chain comprising the amino acid sequence set forth in SEQ ID NO: 1. In certain aspects of the invention, the antibody or antigen-binding fragment thereof comprises a variable light chain comprising the amino acid sequence set forth in SEQ ID NO: 3. In certain aspects of the invention, the antibody or antigen-binding fragment thereof comprises a variable light chain comprising the amino acid sequence set forth in SEQ ID NO: 5. In certain aspects of the invention, the antibody or antigen-binding fragment thereof comprises a variable light chain comprising the amino acid sequence set forth in SEQ ID NO: 7. In certain aspects of the invention, the antibody or antigen-binding fragment thereof comprises a variable heavy chain comprising an amino acid sequence that is at least about 80% homologous or identical to the amino acid sequence set forth in SEQ ID NO: 9. In certain aspects of the invention, the antibody or antigen-binding fragment thereof comprises the amino acid sequence set forth in SEQ ID NO: 9.

In certain aspects of the invention, the antibody or antigen-binding fragment thereof comprises an amino acid sequence that is at least about 80% homologous or identical to the amino acid sequence set forth in SEQ ID NO: 11, SEQ ID NO: 13, SEQ ID NO: 15, or SEQ ID NO: 17. In certain aspects of the invention, the antibody or antigen-binding fragment thereof comprises the amino acid sequence set forth in SEQ ID NO: 11, SEQ ID NO: 13, SEQ ID NO: 15, or SEQ ID NO: 17. In certain aspects of the invention, the antibody or antigen-binding fragment thereof comprises the amino acid sequence set forth in SEQ ID NO: 11. In certain aspects of the invention, the antibody or antigen-binding fragment thereof comprises the amino acid sequence set forth in SEQ ID NO: 13. In certain aspects of the invention, the antibody or antigen-binding fragment thereof comprises the amino acid sequence set forth in SEQ ID NO: 15. In certain aspects of the invention, the antibody or antigen-binding fragment thereof comprises the amino acid sequence set forth in SEQ ID NO: 17.

In certain aspects of the invention, the recombinant surface protein comprises a glycoprotein. In certain aspects of the invention, the glycoprotein comprises an amino acid sequence that is at least about 80% homologous or identical to the amino acid sequence set forth in SEQ ID NO: 21. In certain aspects of the invention, the glycoprotein comprises the amino acid sequence set forth in SEQ ID NO: 21. In certain aspects of the invention, the glycoprotein comprises insertion, deletion, or substitution of one or more amino acid residues. In certain aspects of the invention, the amino acid residue is at position 10, 47, 156, 184, 194, 238, 354, and 397 of SEQ ID NO: 21. In certain aspects of the invention, the glycoprotein comprises:
a) a substitution of a glutamine residue with a lysine residue at position 10 of SEQ ID NO: 21;
b) a substitution of a lysine residue with a glutamine residue at position 47 of SEQ ID NO: 21;
c) a substitution of a tyrosine residue with an aspartic acid residue at position 156 of SEQ ID NO: 21;
d) a substitution of a methionine residue with a threonine residue at position 184 of SEQ ID NO: 21;
e) a substitution of a glutamic acid residue with a lysine residue at position 194 of SEQ ID NO: 21;
f) a substitution of a glutamic acid residue with a lysine residue at position 238 of SEQ ID NO: 21;
g) a substitution of an arginine residue with a glutamine residue at position 354 of SEQ ID NO: 21;
h) a substitution of a histidine residue with an arginine residue at position 397 of SEQ ID NO: 21; or
i) a combination thereof.

In certain aspects of the invention, the glycoprotein comprises a substitution of a glutamine residue with a lysine residue at position 10 of SEQ ID NO: 21 and a substitution of a lysine residue with a glutamine residue at position 47 of SEQ ID NO: 21. In certain aspects of the invention, the glycoprotein comprises an amino acid sequence that is at least about 80% homologous or identical to the amino acid sequence set forth in SEQ ID NO: 23. In certain aspects of the invention, the glycoprotein comprises the amino acid sequence set forth in SEQ ID NO: 23. In certain aspects of the invention, the glycoprotein comprises an amino acid sequence that is at least about 80% homologous or identical to the amino acid sequence set forth in SEQ ID NO: 27. In certain aspects of the invention, the glycoprotein comprises the amino acid sequence set forth in SEQ ID NO: 27.

In certain aspects of the invention, the recombinant target surface molecule comprises a signal sequence. In certain aspects of the invention, the signal sequence comprises from amino acid 1 to amino acid 16 of SEQ ID NO: 19. In certain aspects of the invention, the recombinant target surface molecule comprises, from N-end to C-end, a signal peptide, a first linker, an antibody or antigen-binding fragment thereof, a second linker and a glycoprotein. In certain aspects of the invention, the recombinant target surface molecule is integrated into the genome of the recombinant virus.

Furthermore, the present disclosure provides methods of treating or preventing a subject having disease, disorder, or condition, (e.g., cancer). In certain aspects of the invention, the methods comprise administering to the subject the recombinant virus disclosed here. In certain aspects of the invention, the method improves the anti-cancer adaptive immune response in the subject and/or promotes the immunogenicity of a tumor microenvironment of the subject. In certain aspects of the invention, the cancer is selected from the group consisting of adenocarcinoma, osteosarcoma, cervical carcinoma, melanoma, hepatocellular carcinoma, breast cancer, metastatic breast cancer, lung cancer, prostate cancer, ovarian cancer, leukemia, lymphoma, renal carcinoma, pancreatic cancer, gastric cancer, colon cancer, duodenal cancer, glioblastoma multiforme, astrocytoma, sarcoma, and combinations thereof. In certain aspects of the invention, the cancer is breast cancer or metastatic breast cancer. In certain aspects of the invention, the cancer is prostate cancer. In certain aspects of the invention, the subject is human.

In certain aspects of the invention, the methods further comprise administering to the subject an immunomodulatory agent selected from the group consisting of immune checkpoint inhibitors, T cells, dendritic cells, therapeutic antibodies, cancer vaccines, cancer cell antigens, infectious disease antigens, autoimmune disease antigens, cytokines, Bacillus Calmette-Guerin (BCG), and any combinations thereof. In certain aspects of the invention, the immunomodulatory agent is selected from the group consisting of anti-PD1 antibodies or an antigen-binding fragment thereof, anti-PD-L1 antibodies or an antigen-binding fragment thereof, anti-CTLA-4 antibodies or an antigen-binding fragment thereof, anti-BTLA antibodies or an antigen-binding fragment thereof, anti-TIGIT antibodies or an antigen-binding fragment thereof, anti-TIM3 antibodies or an antigen-binding fragment thereof, anti-LAG-3 antibodies or an antigen-binding fragment thereof, anti-IL-10 or IL-10R antibodies or an antigen-binding fragment thereof, anti-TGF or TGFR antibodies or an antigen-binding fragment thereof and any combinations thereof. In certain aspects, the immunomodulatory agent is a cytokine such as, but not exclusively confined to, GMCSF, TGF, TNF-α, CD40L, FLT3L, IFN-α, IFNβ, IFN-γ, IL-1, IL-2, IL-4, IL-5, IL-6, IL-7, IL-8, IL-12, IL-15, IL-21, IL-23, MIP-1, and MCP-1. Examples of other genes, include, but is not limited to, urokinase, herpesvirus thymidine kinase (HSV-TK), purine nucleoside phosphorylase, cytosine deaminase, and EGFP.

Also disclosed herein are methods for improving a subject's responsiveness to an immunomodulatory agent, comprising administering to the subject the immunomodulatory agent and the recombinant virus disclosed herein, wherein the subject has cancer.

The present disclosure further provides pharmaceutical compositions comprising the recombinant virus disclosed herein. In certain aspects of the invention, the pharmaceutical compositions further comprise an immunomodulatory agent.

### BRIEF DESCRIPTION OF THE DRAWINGS

FIG. 1 depicts the rrVSV-G genome drawn to scale. The VSV-G gene has been replaced with a mutant G-gene that disables the LDL-R binding site and adds SCA-erbb2 to the amino terminus. The gene for EGFP has been added to the genome as annotated.

FIGS. 2A and 2B depict titer on two pairs of cell lines of rrVSV-G-SCA-erbb2 and wtVSV. SKBR3 and D2F2/E2 are cell lines that express erbb2. 143 and D2F2 are cell lines that do not express erbb2. Mean of three experiments with SEM bars.

FIG. 3 depicts inhibition by anti-erbb2 MAb, HERCEPTIN™, of infection by VSV-G on D2F2/E2 cells that express Her2/neu and D2F2 cells that do not. Mean of three experiments with SEM bars. The X-axis is log 2 scale.

FIG. 4 depicts the growth assay and yield of VSV-G-SCA-erbb2 at various times following infection, at an MOI of 0.001, of D2F2/E2 cells that express Her2/neu and D2F2 cells that do not. The titer of the yield was determined on D2F2/E2 cells. Mean of three experiments with SEM bars.

FIG. 5 depicts the growth assay and yield of VSV-G-SCA-erbb2 at various times following infection, at an MOI of 10, of D2F2/E2 cells that express Her2/neu and D2F2 cells that do not. The titer of the yield was determined on D2F2/E2 cells. Mean of three experiments with SEM bars.

FIG. 6 shows the comparison of viral stability in vitro at 37° C. between the VSV-G-SCA-erbb2, VSV-Sindbis-SCA-erbb2, and wtVSV-EGFP. The rate of decay and half-life did not differ significantly among the 3 viruses (t½=4.7 hours for wtVSV, 5.3 hours for rrVSV-Sindbis, and 5.87 hours for rrVSV-G). Non-linear regression one phase decay goodness of fit R2=0.9681 for wtVSV, 0.9464 for rrVSV-Sindbis and 0.9747 for rrVSV-G. Data are means of results from 3 experiments with SEM bars. The X-axis is log 2 scale.

FIG. 7 depicts an in vivo growth assay. Yield of virus at various times following infection of subcutaneous D2F2/E2 or D2F2 tumors. The titer of the yield was determined on D2F2/E2 cells. Area under the Curve (AUC) was significantly higher following infection of D2F2/E2 than D2F2 (unpaired t-test: p=0.03); n=5 for each time point except day 3 which had 3. Mean titers with SEM bars.

FIGS. 8A-8D show histopathological examination of peritoneal tumor nodules at various times after implantation. FIG. 8A depicts a photomicrograph of multiple nodules of day 5 tumors seen above the size bar (1000 μm). H&E ×20. FIG. 8B depicts a photomicrograph of the tumor in FIG. 8A. H&E ×100. FIG. 8C depicts multiple nodules of day 7 tumors seen above the size bar (1000 μm). H&E ×20. FIG. 8D depicts multiple nodules of day 10 tumors seen below the size bar (1000 μm). H&E ×20.

FIGS. 9A and 9B depict survival curves of D2F2/E2 peritoneal implants treated with rrVSV-G alone, rrVSV-Sindbis alone or both. FIG. 9A shows five-day tumors showed better survival following treatment with rrVSV-G than rrVSV-Sindbis (n=10 per group; log rank statistic: p=0.01). FIG. 9B shows seven and 10-day tumors showed better survival following treatment with rrVSV-G than control (n=10 per group; log rank statistic: p<0.0001, for both 7-day and 10-day tumors).

FIG. 10 shows adaptation of rrVSV-G-SCA-erbb2. The table reports the passage number when mutations were first identified and titers on D2F2/E2, a cell line that expresses Her2/neu and D2F2, the parent cell line that does not. Further a schematic of G-SCA compound protein and recombinant inserted mutations (K47Q and R354Q) and adapted mutations is shown. Domains I-IV and transmembrane domain of VSV-G are labelled according to the crystal structure (Roche, et al., Science, 313: 187-91 (2007)). All numbering of amino acids described herein follow the scheme used in this figure. The signal sequence, the first linker-SCA-second linker domain and the G glycoprotein domain each have their own numbering system, beginning with 1.

FIG. 11 shows location of rrVSV-G mutations on the crystallographic structure of the SCA and VSV-G. Mutations T91 and T93 were labeled on the SCA using the template from PDB (PDB ID: 6ZQK. HER2-binding scFv-Fab fusion 841). As shown, both mutations are not located at the hypervariable region. The conformation of VSV-G in complex with Low-density lipoprotein receptor (LDL-R) is derived from VSV G CR2 (PDB ID: 5OYL). Two residues, K47 and R354, essential for the natural binding of VSV-G, were mutated to Q. All six mutations created during adaptation are labeled using balls. A sphere represents a large insertion of SCA segment at the beginning of VSV G.

FIG. 12 shows a set of flow cytometry plots following analysis of peritoneal cells harvested from animals (“Mouse 1,” “Mouse 2,” “Mouse 3,” “Mouse 4,” and “Mouse 5”) 5 days after intraperitoneal (IP) D2F2/E2 cell challenge. Cured animals were challenged with i.p. D2F2/E2 cells and peritoneal cells were harvested 5 days later. Antitumor memory CD8 T cells were identified by flow cytometry analyses following staining with a tetramer displaying the immunodominant p63 epitope of the Her2/neu protein.

FIG. 13 shows a graph showing a titer of rrVSV-G-SCA-PSMA, a virus of an aspect of the invention, and wild type VSV (wtVSV) on PSMA positive (PC3-PIP) and parent negative cell lines (PC-3). The mean of three experiments with SEM bars are shown.

FIG. 14 shows a graph showing the yield of VSV-G-SCA-PSMA, a virus of an aspect of the invention, at various times following low MOI infection (MOI=0.01) of a parent cell line PC3 that does not express PSMA and the same cells stably transfected to express PSMA (PC3-PIP). The titer of the yield was determined on PC3-PIP cells. Shown are the means of three experiments with SEM bars. AUC 95% confidence intervals are as follows: PC3-PIP: 5.43E+07 to 8.47E+07 and PC3: 1.06E+05 to 1.83E+05.

FIG. 15 shows a graph showing survival curves of D2F2/E2 peritoneal implants treated IV or IP with rrVSV-G-SCA-erbb2, a virus of an aspect of the invention, or with control treatment without virus. Both IP and IV viral treatment showed significantly better survival than control treatment (log rank statistic: p<0.05). At the 50 hour mark, the top line is IP, the middle line is IV, and the bottom line is the control. Cure was achieved in 75% of the animals treated IP, 50% of those treated IV and none of the controls.

### DETAILED DESCRIPTION

Viruses and viral vectors are being designed for cancer therapy and tested in multiple clinical trials. Safety is a critical issue in patients who are often immunologically compromised. Targeting the virus to specific cells (e.g., cancer cells) improves safety and can be achieved in several ways, including specifying the binding between the virus and the target cell entry receptors, deleting essential viral genes that are required for viral replication in normal cells, or genetically modifying the virus to require tumor-specific promoters for viral selective transcription.

VSV is an excellent candidate for development as an oncolytic virus because it is an efficient cell killer that does not produce serious human disease and has no risk of host cell transformation. Normal tissues are protected from the virus by interferon (IFN) production but most human tumors are insensitive to the effects of IFN and susceptible to killing by VSV. Rodent studies have shown the potential of neurotoxicity with the use of wild-type (wt) VSV leading to the development of recombinant attenuated VSV. Currently, Phase 1 clinical trials are using VSV-encoding IFNβ (VSV-IFNβ), to treat various cancers. An aspect of the invention provides novel approaches for developing a clinically useful VSV oncolytic virus and a platform for developing any targeted replicating or non-replicating virus using the glycoprotein disclosed herein.

rrVSV-C is a new targeted oncolytic virus that has significant advantages over current viruses used for cancer therapy. It is safe because it is highly targeted to a specific cancer cell type, very poorly infects normal cells, and grows to high titer. It is more potent than other oncolytic viruses because it consistently cures established tumors in pre-clinical mouse models. It generates a therapeutic anti-tumor immune response. It also uses the native VSV-G glycoprotein which allows for heterologous combination with any other oncolytic virus that has a different glycoprotein.

rrVSV-G was created by altering the VSV genome to express one chimeric surface glycoprotein combining a single chain antibody (SCA) recognizing the HER2/neu receptor with the VSV-G protein. Native binding of VSV-G was reduced by 2 mutations, K47Q and R354Q Targeting of the SCA was improved by 2 mutations (see U.S. Pat. No. 7,429,481). The virus was further improved as disclosed herein as aspects of the invention, and can include one or more of the following:
Creation of a new replicating targeted virus expressing a new chimeric surface glycoprotein as described herein;
Creation of a new method to generate targeting surface glycoproteins that can be used to replace the native glycoprotein on any replicating or non-replicating virus, as described herein;
The choice of 2 specific linkers to create the chimeric SCA-G protein (e.g., a first linker and a second linker);
The insertion of the “first linker-SCA-second linker” combination at a particular location in the G protein;
P278L mutation in the second linker;
T91N mutation in the V_{L} region of the SCA;
T93N mutation in the V_{L} region of the SCA;
Q10K mutation in the VSV-G protein;
Y156D mutation in the VSV-G protein;
M184T mutation in the VSV-G protein;
E194K mutation in the VSV-G protein;
E238K mutation in the VSV-G protein; and
H397R mutation in the VSV-G protein.

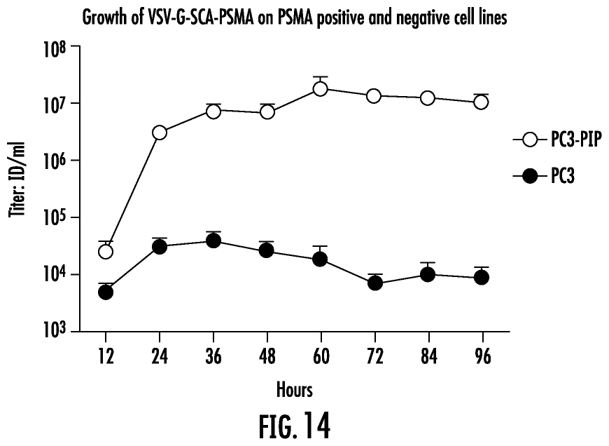

rrVSV-G has a more generalized applicability than only treatment of HER2/neu breast cancer. The current SCA-HER2/neu can be easily replaced with SCAs targeting different cells to use viral lysis and immune stimulation to treat a wide variety of neoplastic, infectious, and autoimmune diseases. For example, rrVSV-G has been used to treat prostate cancer in mouse model (see Example 2 and FIGS. 13 and 14), rrVSV-G is an immune stimulant and can therefore be used in a vaccine strategy as well as a treatment one. There is also room in the viral genome to add genes expressing cytokines or immune modulators or cancer cell antigens, infectious disease antigens, or autoimmune disease antigens. The chimeric glycoprotein developed as described herein can be used to replace the native glycoprotein on any replicating or non-replicating virus and create a non-VSV targeted virus.

Non-limiting aspects of the invention are described by the present specification and Examples.

For purposes of clarity of disclosure and not by way of limitation, the detailed description is divided into the following subsections:
1. Definitions;
2. Target Surface Molecules;
3. Recombinant Viruses;
4. Pharmaceutical Compositions;
5. Methods of Prevention and Treatment; and
6. Kits.

### 1. Definitions

The terms used in this specification generally have their ordinary meanings in the art, within the context of this disclosure, and in the specific context where each term is used. Certain terms are discussed below, or elsewhere in the specification, to provide additional guidance to the practitioner in describing the compositions and methods of the disclosure and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one skill in the art is cognizant that these terms are open-ended terms.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other aspects “comprising,” “consisting of”, and “consisting essentially of,” the aspects or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “nucleic acid molecule” and “nucleotide sequence,” as used herein, refers to a single or double-stranded covalently-linked sequence of nucleotides in which the 3′ and 5′ ends on each nucleotide are joined by phosphodiester bonds. The nucleic acid molecule can include deoxyribonucleotide bases or ribonucleotide bases and can be manufactured synthetically in vitro or isolated from natural sources.

The terms “polypeptide,” “peptide,” “amino acid sequence” and “protein,” used interchangeably herein, refer to a molecule formed from the linking of at least two amino acids. The link between one amino acid residue and the next is an amide bond and is sometimes referred to as a peptide bond. A polypeptide can be obtained by a suitable method known in the art, including isolation from natural sources, expression in a recombinant expression system, chemical synthesis, or enzymatic synthesis. The terms can apply to amino acid polymers in which one or more amino acid residues is an artificial chemical mimetic of a corresponding naturally occurring amino acid, as well as to naturally occurring amino acid polymers and non-naturally occurring amino acid polymers.

As used herein, the term “mutation” refers to a mutation in an amino acid sequence or a nucleic acid sequence. In certain aspects, a mutation in an amino acid sequence can be a substitution (replacement), an insertion (addition), or a deletion (truncation) of at least one amino acid in the amino acid sequence. In certain aspects, a mutation in a nucleic acid sequence can be a substitution (replacement), an insertion (addition), or a deletion (truncation) of at least a nucleotide of the nucleic acid sequence.

As used herein, the term “antibody or fragments thereof” encompasses chimeric antibodies and hybrid antibodies, with dual or multiple antigens or epitope specificities, and fragments, such as F(ab′)2, Fab′, Fab, Fv, scFv, camelid-like antibodies, and the like, including hybrid fragments. Thus, fragments of the antibodies that retain the ability to bind their specific antigens are provided. For example, fragments of antibodies that maintain target binding activity are included within the meaning of the term “antibody or fragment thereof.” Methods for producing antibodies and screening antibodies for specificity and activity are described in Harlow and Lane. Antibodies, A Laboratory Manual. Cold Spring Harbor Publications, New York, (1988). Also included within the meaning of “antibody or fragments thereof” are conjugates of antibody fragments and antigen binding proteins (single chain antibodies). The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional properties, such as removing/adding amino acids capable of disulfide bonding, increasing its bio-longevity, altering its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment (Zoller, et al., Cure. Opin. Biotechnol., 3: 348-354, (1992)).

The term “endogenous,” as used herein, refers to a nucleic acid molecule or polypeptide that is normally expressed in a cell or tissue.

The term “exogenous,” as used herein, refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By “increase” is meant to alter positively by at least about 5%. An alteration can be an increase of about 5%, about 10%, about 25%, about 30%, about 50%, about 75%, about 100% or more.

By “reduce” is meant to alter negatively by at least about 5%. An alteration can be a decrease of about 5%, about 10%, about 25%, about 30%, about 50%, about 75% or more, even by about 100%.

As used herein, “a functional fragment” of a molecule or polypeptide includes a fragment of the molecule or polypeptide that retains at least about 80%, at least about 85%, at least about 90%, at least about 95%, or at least about 100% of the primary function of the molecule or polypeptide.

As used herein, the term “substantially identical” or “substantially homologous” refers to a polypeptide or a nucleic acid molecule exhibiting at least about 50% identical or homologous to a reference amino acid sequence (for example, any of the amino acid sequences described herein) or a reference nucleic acid sequence (for example, any of the nucleic acid sequences described herein). In certain aspects of the invention, such a sequence is at least about 60%, at least about 65%, at least about 70%, at least about 75%, at least about 80%, at least about 85%, at least about 90%, at least about 95%, at least about 99%, or at least about 100% identical or homologous to the amino acid sequence or the nucleic acid sequence used for comparison.

An “individual” or “subject” herein is a vertebrate, such as a human or non-human animal, for example, a mammal. Mammals include, but are not limited to, humans, non-human primates, farm animals, sport animals, rodents, and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “infection” refers to a condition caused by an infectious pathogen, e.g., virus, bacteria, fungus, or parasite.

As used herein, the term “therapeutically effective amount” or “effective amount” refers to an amount of a recombinant virus of an aspect of the invention (e.g., a VSV), or a pharmaceutical composition comprising a recombinant virus of an aspect of the invention, that is sufficient to reduce, inhibit, or abrogate a disease, condition, or disorder (e.g., tumor cell growth), in vitro or in vivo. In certain aspects of the invention, the reduction, inhibition, or abrogation of tumor cell growth may be the result of an immune response, necrosis, apoptosis, or other forms of programmed cell death, as in pyroptosis, paraptosis, ferroptosis, PANoptosis, NEToptosis, phagoptosis, entosis, autosis, autophagic cell death, mitotic catastrophe, and immunogenic cell death. The amount of a recombinant virus of an aspect of the invention (e.g., a VSV), or a pharmaceutical composition comprising a recombinant virus of an aspect of the invention, that is therapeutically effective or effective may vary depending on the context. An effective amount can be administered in one or more administrations.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more signs or symptoms, diminishment of the extent of disease, stabilized (i.e., not worsening) state of disease, prevention of disease, delay or slowing of disease progression, and/or amelioration or palliation of the disease state. The decrease can be about 10%, about 20%, about 30%, about 40%, about 50%, about 60%, about 70%, about 80%, about 90%, about 95%, about 98% or about 99% decrease in severity of complications or symptoms. “Treatment” can also mean prolonging survival as compared to expected survival if not receiving treatment. “Treatment” can also mean prevention of a deleterious outcome. One, non-exclusive, example is vaccination leading to prevention of cancer development, cancer growth, cancer spread, cancer recurrence, and/or cancer metastases. One, non-exclusive, mechanism is generation of memory T and/or B lymphocytes.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part that is not directly connected to the organ of the original cancerous tumor. Metastasis can also be defined as several steps of a process, such as the departure of cancer cells from an original tumor site, and migration and/or invasion of cancer cells to other parts of the body.

As used herein, the phrase “autoimmune disorder” refers to means a disease or disorder caused by a reaction produced by the body against its own tissues or organs or cosegregation or manifestation of these disorders or condition associated with them.

“In combination with,” as used herein, means that a recombinant virus of an aspect of the invention (e.g., a VSV), and one or more agents, e.g., an immunomodulatory agent, are administered to a subject as part of a treatment regimen or plan.

### 2. Target Surface Molecules

An aspect of the invention provides recombinant viruses (e.g., VSV) comprising target surface molecules including proteins, glycoproteins, glycolipids, carbohydrates and lipids that are associated with a disease, disorder, or condition. The disease, disorder, or condition includes, for example, an infectious disease, an autoimmune disorder, and cancers. In an aspect, the target surface molecule is a target surface protein.

An aspect of the invention provides recombinant viruses (e.g., VSV) comprising target surface molecules including proteins, glycoproteins, glycolipids, carbohydrates and lipids that are associated with an infectious disease (i.e., a disease or disorder caused by a bacterium, a virus, a fungus, or a parasite). Examples of infectious diseases that can be treated or prevented by the inventive methods include, but are not limited to, diseases caused by a human immunodeficiency virus (HIV), human papillomavirus, a respiratory syncytial virus (RSV), an influenza virus, a dengue virus, Epstein-Barr virus, a hepatitis B virus (HBV), a hepatitis C virus (HCV)), or a member of the Coronaviridae family (e.g., Severe Acute Respiratory Syndrome (SARS), Coronavirus and Coronavirus of the Middle East Respiratory Syndrome (MERS), tuberculosis and malaria. Examples of target molecules including proteins, glycoproteins, glycolipids, carbohydrates and lipids that are associated with an infectious disease include Tb Antigens 85A/B, ESAT-6, CFD-10, Malaria AMA1, EBA-175 and rCSF and the surface glycoproteins of human immunodeficiency virus (HIV), respiratory syncytial virus (RSV), influenza virus, dengue virus, Epstein-Barr virus, hepatitis B virus (HBV), hepatitis C virus (HCV)), or a member of the Coronaviridae family.

As aspect of the invention provides recombinant viruses (e.g., VSV) comprising target surface molecules including proteins, glycoproteins, glycolipids, carbohydrates and lipids that are associated with an “autoimmune disorder” (i.e., a disease or disorder caused by a reaction produced by the body against its own tissues or organs or cosegregation or manifestation of these disorders or condition associated with them). Examples of target molecules including proteins, glycoproteins, glycolipids, carbohydrates and lipids that are associated with an “autoimmune disorder” include thyroid-stimulating hormone, thyroid peroxidase, insulin, chromatin, spliceosome complex and the MOG, NMO, and NMDA receptors. As aspect of the invention provides recombinant viruses (e.g., VSV) comprising target surface molecules including proteins, glycoproteins, glycolipids, carbohydrates and lipids that are associated with cancer. For example, the target surface molecules including proteins, glycoproteins, glycolipids, carbohydrates and lipids that are associated with cancer can include HER2, CD9, CD20, CD33, TRP1/gp75, gp100, CEA, EGFR3, EGFRvIII, EGFR, GD2, Muc-1, PMSA and EphA3. Examples of internal cancer proteins that can be expressed by the invented virus include p53, BRCA1, BRCA2, β-catenin, CDK4, WT-1 and NY-ESO-1.

An aspect of the invention provides recombinant viruses (e.g., VSV) targeting HER2. The HER2 receptor (also known as c-erbB-2, HER2/neu, human EGF receptor 2, and human epidermal growth factor receptor 2) is a transmembrane glycoprotein with tyrosine kinase activity. Aberrant HER2 protein overexpression has associations with some adenocarcinomas, including breast, ovary, endometrium, cervix as well as lung, esophageal, gastroesophageal junction, gastric, and bladder cancers. HER2 is associated with increased disease recurrence and is a poor prognostic factor for survival.

In pathophysiological situations, overexpression of HER2 is mainly associated with HER2 gene amplification and results in constitutive activation of the HER2 downstream signaling pathway (Yarden & Sliwkowski, Nat. Rev. Mol. Cell Biol., 2(2): 127-37 (2001)). Activation of HER2 pathways induced homodimerization which leads to phosphorylation of tyrosine residues, and activation of downstream signaling including PI3K/AKT signaling cascade, Ras/MEK/ERK, and JAK/STAT. These pathways regulate cell survival, proliferation, differentiation, motility, apoptosis, survival, invasion, migration, adhesion, and angiogenesis (Yarden & Sliwkowski, Nat. Rev. Mol. Cell Biol., 2(2): 127-37 (2001)).

HER2 amplification is a responsible for human breast tumorigenesis (Mamani-Cancino et al., Ginecol. Obstet. Mex., 82(6): 369-76 (2014)). HER2-positive breast cancers have unique biological and clinical characteristics such as increased proliferation rates, high histologic and nuclear grade, low ER and PR levels, more aneuploidy, and tendency to metastasize to CNS viscera, relative resistance to endocrine therapy, and increased sensitivity to doxorubicin. Patients having HER2 positive metastatic breast cancer are traditionally treated with a combination of chemotherapy and HER2-directed agent (e.g., monoclonal antibodies). Patients with hormone receptor and HER2-positive metastatic breast cancer can also receive a combination of HER2 directed therapy (e.g., monoclonal antibodies) and an endocrine therapy.

An aspect of the invention provides recombinant viruses (e.g., VSV) targeting PSMA. PSMA is a transmembrane glycoprotein that is nearly universally expressed in prostate cancer.

### 3. Recombinant Viruses

An aspect of the invention provides recombinant viruses comprising target surface molecules. In certain aspects of the invention, the viruses are enveloped viruses or non-replicating pseudoviruses. Non-limiting examples of enveloped viruses include vesicular stomatitis virus (“VSV”), or members of the Retrovirus, Rhabdovirus, Alphavirus, Togavirus, Flavivirus, Coronavirus, Orthomyxovirus, or Bunyavirus family. In certain aspects of the invention, the enveloped virus is a vesicular stomatitis virus. In certain aspects, the recombinant virus is an enveloped virus. In another aspect, the recombinant virus is a non-enveloped virus. In an aspect, the recombinant is an RNA enveloped virus. In an aspect, the recombinant virus is a negative stranded RNA envelope virus. In another aspect, the recombinant virus is a positive stranded RNA envelope virus.

### Replicating or Non-Replicating

In certain aspects of the invention, the genome of a recombinant virus disclosed herein includes a site for insertion of a surface protein associated with infectivity. In certain aspects of the invention, the genome of a recombinant virus disclosed herein includes a site for insertion of a therapeutic gene (e.g., a cytokine, cytokine agonist, or cytokine antagonist). In certain aspects of the invention, the genome of a recombinant virus disclosed herein includes a site for insertion of a surface protein associated with infectivity and a therapeutic gene (e.g., a cytokine, cytokine agonist, or cytokine antagonist). In certain non-limiting aspects of the invention, each site can be flanked by restriction enzyme cleavage sites to facilitate the insertion of cassettes comprising the desired genes (e.g., surface protein associated with infectivity). In certain aspects of the invention, the inserted genes can be expressed according to the life cycle of the virus (see below for “stop-start” site for VSV), and where appropriate should be operably linked to a suitable promoter element.

In certain aspects of the invention, the surface protein associated with infectivity comprises an IgG-binding domain. For example, but without any limitation, the IgG-binding domain is a polypeptide derived from staphylococcal protein A that bind to the Fc (constant) region of IgG immunoglobulins. In certain aspects of the invention, the display of a synthetic derivative of IgG-binding domains of protein A (ZZ) domains promotes targeting the recombinant VSV to the host cell of interest when used in combination with an appropriate antibody. In certain aspects of the invention, this ZZ modified VSV can be targeted to any antigen by using the appropriate antibody. In certain aspects of the invention, the antibody targets cell surface markers of cancer cells, specific cells of the immune system, or any cell with a relatively specific cell surface marker in order to delete that cell.

In certain aspects of the invention, the viral target surface molecule associated with infectivity comprises a ligand that binds to a cellular receptor. For example, but without any limitation, the ligand can bind to transferrin, a cytokine (such as ciliary neurotrophic factor) or a receptor-binding antagonist thereof, ligands for integrin or cadherin molecules, etc.

In certain aspects of the invention, the recombinant virus includes a target surface molecule associated with infectivity. In certain aspects of the invention, the target surface molecule associated with infectivity comprises an antibody or antigen-binding fragment thereof. In certain aspects of the invention, the antibody or antigen-binding fragment thereof is an scFv or a single-chain antibody (SCA) with specificity for a cell surface protein. The antibody or antigen-binding fragment thereof can target the virus to any cell surface molecule including proteins, glycoproteins, glycolipids, carbohydrates, and lipids. For example, but without any limitation, the antibody or antigen-binding fragment thereof can target p53, Ras, β-catenin, CDK4, CDC27, α actinin-4, HER2, WT1, EphA3, EGFR, CD4, CD8, CD20, MAGE, BAGE, GAGE, NY-ESO-1, Tyrosinase, TRP1/gp75, TRP2, gp100, Melan-A/MART1, gangliosides, or PSMA.

In certain aspects of the invention, the antibody or antigen-binding fragment thereof can target HER2. In certain aspects of the invention, the antibody or antigen-binding fragment thereof targeting HER2 is human. In certain aspects, of the invention the antibody or antigen-binding fragment thereof targeting HER2 is humanized. In certain aspects, of the invention the antibody or antigen-binding fragment thereof targeting HER2 is murine. In certain aspects of the invention, the antibody or antigen-binding fragment thereof is derived from pertuzumab. In certain aspects of the invention, the antibody or antigen-binding fragment thereof is derived from trastuzumab. Trastuzumab is a recombinant IgG1 kappa, humanized monoclonal antibody that selectively binds with high affinity in a cell-based assay (Kd=5 nM) to the extracellular domain of HER2. Trastuzumab is used as a treatment of HER2⁺ metastatic breast cancer, where there is a proven amplification of the HER2 oncogene or over-expression of the HER2 protein in tumors. Trastuzumab can bind to HER2 and suppresses cancer cell growth, proliferation, and survival directly and indirectly. A non-exclusive list of antibodies or antibody fragments that target HER2 and could be used in an aspect of the invention includes the following:
JP 2017518038-A/8: Mutated Antibody of Fully Humanized HER2 Antibody, and Encoding Gene and use thereof, Genbank Accession: LZ215386.1, GI: 1287612721;
KR 1020160145185-A/15: Mutated Antibody of Fully Human HER2 Antibody, and encoding gene and use thereof, Genbank Accession: LY468885.1, GI: 1258059152;
JP 2021531802-A/9: Antibody Binding to Human HER2 and Preparation Method and Use Thereof, Genbank Accession: PA369638.1, GI: 2214705484;
KR 1020200122423-A/80: Site Specific HER2 Antibody Drug Conjugates, Genbank Accession: OF120327.1, GI: 2077191655;
JP 2018537975-A/36: Site Specific HER2 Antibody Drug Conjugates, Genbank Accession: MC035558.1, GI: 1885295312;
JP 2013531474-A/5: Lytic-Peptide-Her2/neu (Human Epidermal growth factor Receptor 2) Ligand Conjugates and Methods of Use, Genbank Accession: HW328313.1, GI: 568766807;
KR 1020140119318-A/110: Bispecific anti-cMet/anti-Her2 antibodies, Genbank Accession: DI434027.1, GI: 930906588;
WO 2020196475-A/12: Anti-HER2 antibody-Pyrrolobenzodiazepine Derivatives Conjugate, Genbank Accession: OG158992.1, GI: 1995280442;
KR 1020150000797-A/67: Protein complex, bispecific anti-c-MET/Her2 antibody comprising the protein complex, and method of preparation thereof, Genbank Accession: D1489177.1, GI: 972735474;
Synthetic construct clone P1h3 single chain variable fragment antibody gene, complete cds, Accession: KY670784.1, GI: 1339868116 (all incorporated by reference herein).

In certain aspects of the invention, the antibody or antigen-binding fragment thereof comprises a variable light chain. In certain aspects of the invention, the variable light chain comprises an amino acid sequence that is at least about 80%, at least about 85%, at least about 90%, at least about 95%, at least about 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous to the amino acid sequence set forth in SEQ ID NO: 1. In certain aspects of the invention, the variable light chain comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) identical to the amino acid sequence set forth in SEQ ID NO: 1. In certain aspects of the invention, the variable light chain comprises the amino acid sequence set forth in SEQ ID NO: 1 or a conservative amino acid substitution thereof. In certain aspects, the variable light chain comprises the amino acid sequence set forth in SEQ ID NO: 1. In certain aspects of the invention, the variable light chain consists of the amino acid sequence set forth in SEQ ID NO: 1. SEQ ID NO: 1 is provided below:

DIQMTQSPSSLSASVGDRVTITCRASQDVNTAVAWYQQKPGKAPK
LLIYSASFLYSGVPSRFSGSRSGTDFTLTISSLQPEDFATYYCQQ
HYTTPPTFGQGTKVEIK

In certain aspects, the variable light chain is encoded by a nucleotide sequence set forth in SEQ ID NO: 2, which is provided below:

gatatccagatgacccagtccccgagctccctgtccgcctctgtg
ggcgatagggtcaccatcacctgccgtgccagtcaggatgtgaat
actgctgtagcctggtatcaacagaaaccaggaaaagctccgaaa
ctactgatttactcggcatccttcctctactctggagtcccttct
cgcttctctggatccagatctgggacggatttcactctgaccatc
agcagtctgcagccggaagacttcgcaacttattactgtcagcaa
cattatactactcctcccacgttcggacagggtaccaaggtggag
atcaaa

In certain aspects, the variable light chain comprises an amino substitution of an O-glycosylation site. In certain aspects of the invention, the variable light chain comprises an amino substitution creating an N-glycosylation site. In certain aspects of the invention, the variable light chain comprises an amino acid sequence that is at least about 80%, at least about 85%, at least about 90%, at least about 95%, at least about 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous to the amino acid sequence set forth in SEQ ID NO: 3. In certain aspects of the invention, the variable light chain comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) identical to the amino acid sequence set forth in SEQ ID NO: 3. In certain aspects of the invention, the variable light chain comprises the amino acid sequence set forth in SEQ ID NO: 3 or a conservative amino acid substitution thereof. In certain aspects of the invention, the variable light chain comprises the amino acid sequence set forth in SEQ ID NO: 3. In certain aspects of the invention, the variable light chain consists of the amino acid sequence set forth in SEQ ID NO: 3. SEQ ID NO: 3 is provided below:

DIQMTQSPSSLSASVGDRVTITCRASQDVNTAVAWYQQKPGKAPK
LLIYSASFLYSGVPSRFSGSRSGTDFNLTISSLQPEDFATYYCQQ
HYNTPPTFGQGTKVEIK

In certain aspects, the variable light chain set forth in SEQ ID NO: 3 is encoded by a nucleotide sequence set forth in SEQ ID NO: 4, which is provided below:

gatatccagatgacccagtccccgagctccctgtccgcctctgtg
ggcgatagggtcaccatcacctgccgtgccagtcaggatgtgaat
actgctgtagcctggtatcaacagaaaccaggaaaagctccgaaa
ctactgatttactcggcatccttcctctactctggagtcccttct
cgcttctctggatccagatctgggacggatttcaatctgaccatc
agcagtctgcagccggaagacttcgcaacttattactgtcagcaa
cattatactactcctcccacgttcggacagggtaccaaggtggag
atcaaa

In certain aspects of the invention, the variable light chain comprises an amino substitution of an O-glycosylation site. In certain aspects of the invention, the variable light chain comprises an amino substitution creating an N-glycosylation site. In certain aspects of the invention, the variable light chain comprises an amino acid sequence that is at least about 80%, at least about 85%, at least about 90%, at least about 95%, at least about 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous to the amino acid sequence set forth in SEQ ID NO: 5. In certain aspects of the invention, the variable light chain comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) identical to the amino acid sequence set forth in SEQ ID NO: 5. In certain aspects of the invention, the variable light chain comprises the amino acid sequence set forth in SEQ ID NO: 5 or a conservative amino acid substitution thereof. In certain aspects of the invention, the variable light chain comprises the amino acid sequence set forth in SEQ ID NO: 5. In certain aspects of the invention, the variable light chain consists of the amino acid sequence set forth in SEQ ID NO: 5. SEQ ID NO: 5 is provided below:

DIQMTQSPSSLSASVGDRVTITCRASQDVNTAVAWYQQKPGKAPK
LLIYSASFLYSGVPSRFSGSRSGTDFTLNISSLOPEDFATYYCQQ
HYTTPPTFGQGTKVEIK

In certain aspects of the invention, the variable light chain set forth in SEQ ID NO: 5 is encoded by a nucleotide sequence set forth in SEQ ID NO: 6, which is provided below:

gatatccagatgacccagtccccgagctccctgtccgcctctgtg
ggcgatagggtcaccatcacctgccgtgccagtcaggatgtgaat
actgctgtagcctggtatcaacagaaaccaggaaaagctccgaaa
ctactgatttactcggcatccttcctctactctggagtcccttct
cgcttctctggatccagatctgggacggatttcactctgaacatc
agcagtctgcagccggaagacttcgcaacttattactgtcagcaa
cattatactactcctcccacgttcggacagggtaccaaggtggag
atcaaa

In certain aspects of the invention, the variable light chain comprises an amino substitution of a first O-glycosylation site and a second O-glycosylation site. In certain aspects of the invention, the variable light chain comprises a first amino substitution creating a N-glycosylation site and a second amino substitution creating a N-glycosylation site. In certain aspects of the invention, the variable light chain comprises an amino acid sequence that is at least about 80%, at least about 85%, at least about 90%, at least about 95%, at least about 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous to the amino acid sequence set forth in SEQ ID NO: 7. In certain aspects of the invention, the variable light chain comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) identical to the amino acid sequence set forth in SEQ ID NO: 7. In certain aspects of the invention, the variable light chain comprises the amino acid sequence set forth in SEQ ID NO: 7 or a conservative amino acid substitution thereof. In certain aspects of the invention, the variable light chain comprises the amino acid sequence set forth in SEQ ID NO: 7. In certain aspects of the invention, the variable light chain consists of the amino acid sequence set forth in SEQ ID NO: 7. SEQ ID NO: 7 is provided below:

DIQMTQSPSSLSASVGDRVTITCRASQDVNTAVAWYQQKPGKAPK
LLIYSASFLYSGVPSRFSGSRSGTDFNLNISSLQPEDFATYYCQQ
HYNTPPTFGQGTKVEIK

In certain aspects of the invention, the variable light chain set forth in SEQ ID NO: 7 is encoded by a nucleotide sequence set forth in SEQ ID NO: 8, which is provided below:

gatatccagatgacccagtccccgagctccctgtccgcctctgtg
ggcgatagggtcaccatcacctgccgtgccagtcaggatgtgaat
actgctgtagcctggtatcaacagaaaccaggaaaagctccgaaa
ctactgatttactcggcatccttcctctactctggagtcccttct
cgcttctctggatccagatctgggacggatttcaatctgaacatc
agcagtctgcagccggaagacttcgcaacttattactgtcagcaa
cattatactactcctcccacgttcggacagggtaccaaggtggag
atcaaa

In certain aspects of the invention, the antibody or antigen-binding fragment thereof comprises a variable heavy chain. In certain aspects of the invention, the variable heavy chain comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous to the amino acid sequence set forth in SEQ ID NO: 9. In certain aspects of the invention, the variable heavy chain comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) identical to the amino acid sequence set forth in SEQ ID NO: 9. In certain aspects of the invention, the variable heavy chain comprises the amino acid sequence set forth in SEQ ID NO: 9 or a conservative amino acid substitution thereof. In certain aspects of the invention, the variable heavy chain comprises the amino acid sequence set forth in SEQ ID NO: 9. In certain aspects of the invention, the variable heavy chain consists of the amino acid sequence set forth in SEQ ID NO: 9. SEQ ID NO: 9 is provided below:

EVQLVESGGGLVQPGGSLRLSCAASGFNIKDTYIHWVRQAPGKGL
EWVARIYPTNGYTRYADSVKGRFTISADTSKNTAYLQMNSLRAED
TAVYYCSRWGGDGFYAMDYWGQGTLVTVSSRAA

In certain aspects, the variable heavy chain set forth in SEQ ID NO: 9 is encoded by a nucleotide sequence set forth in SEQ ID NO: 10, which is provided below:

GAGGTTCAGCTGGTGGAGTCTGGCGGTGGCCTGGTGCAGCCAGGG
GGCTCACTCCGTTTGTCCTGTGCAGCTTCTGGCTTCAACATTAAA
GACACCTATATACACTGGGTGCGTCAGGCCCCGGGTAAGGGCCTG
GAATGGGTTGCAAGGATTTATCCTACGAATGGTTATACTAGATAT
GCCGATAGCGTCAAGGGCCGTTTCACTATAAGCGCAGACACATCC
AAAAACACAGCCTACCTGCAGATGAACAGCCTGCGTGCTGAGGAC
ACTGCCGTCTATTATTGTTCTAGATGGGGAGGGGACGGCTTCTAT
GCTATGGACTACTGGGGTCAAGGAACCCTGGTCACCGTCTCCTCG
CGTGCGGCC

In certain aspects of the invention, the antibody or antigen-binding fragment thereof comprises a linker peptide that connects the variable light chain and the variable heavy chain. Any suitable linker peptide known by one of skill in the art can be used.

In certain aspects of the invention, the antibody or antigen-binding fragment thereof comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous to the amino acid sequence set forth in SEQ ID NO: 11. In certain aspects of the invention, the antibody or antigen-binding fragment thereof comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) identical to the amino acid sequence set forth in SEQ ID NO: 11. In certain aspects of the invention, the antibody or antigen-binding fragment thereof comprises the amino acid sequence set forth in SEQ ID NO: 11 or a conservative amino acid substitution thereof. In certain aspects of the invention, the antibody or antigen-binding fragment thereof comprises the amino acid sequence set forth in SEQ ID NO: 11 In certain aspects of the invention, the antibody or antigen-binding fragment thereof consists of the amino acid sequence set forth in SEQ ID NO: 11. SEQ ID NO: 11 is provided below:

DIQMTQSPSSLSASVGDRVTITCRASQDVNTAVAWYQQKPGKAPK
LLIYSASFLYSGVPSRFSGSRSGTDFTLTISSLQPEDFATYYCQQ
HYTTPPTFGQGTKVEIKGGGGSGGGGSGGGGSEVQLVESGGGLVQ
PGGSLRLSCAASGFNIKDTYIHWVRQAPGKGLEWVARIYPTNGYT
RYADSVKGRFTISADTSKNTAYLQMNSLRAEDTAVYYCSRWGGDG
FYAMDYWGQGTLVTVSSRAA

In certain aspects of the invention, the antibody or antigen-binding fragment thereof set forth in SEQ ID NO: 11 is encoded by a nucleotide sequence set forth in SEQ ID NO: 12, which is provided below:

GATATCCAGATGACCCAGTCCCCGAGCTCCCTGTCCGCCTCTGTG
GGCGATAGGGTCACCATCACCTGCCGTGCCAGTCAGGATGTGAAT
ACTGCTGTAGCCTGGTATCAACAGAAACCAGGAAAAGCTCCGAAA
CTACTGATTTACTCGGCATCCTTCCTCTACTCTGGAGTCCCTTCT
CGCTTCTCTGGATCCAGATCTGGGACGGATTTCACTCTGACCATC
AGCAGTCTGCAGCCGGAAGACTTCGCAACTTATTACTGTCAGCAA
CATTATACTACTCCTCCCACGTTCGGACAGGGTACCAAGGTGGAG
ATCAAAGGTGGAGGTGGATCAGGTGGAGGTGGATCAGGTGGAGGT
GGATCAGAGGTTCAGCTGGTGGAGTCTGGCGGTGGCCTGGTGCAG
CCAGGGGGCTCACTCCGTTTGTCCTGTGCAGCTTCTGGCTTCAAC
ATTAAAGACACCTATATACACTGGGTGCGTCAGGCCCCGGGTAAG
GGCCTGGAATGGGTTGCAAGGATTTATCCTACGAATGGTTATACT
AGATATGCCGATAGCGTCAAGGGCCGTTTCACTATAAGCGCAGAC
ACATCCAAAAACACAGCCTACCTGCAGATGAACAGCCTGCGTGCT
GAGGACACTGCCGTCTATTATTGTTCTAGATGGGGAGGGGACGGC
TTCTATGCTATGGACTACTGGGGTCAAGGAACCCTGGTCACCGTC
TCCTCGCGTGCGGCC

In certain aspects of the invention, the antibody or antigen-binding fragment thereof comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous to the amino acid sequence set forth in SEQ ID NO: 13. In certain aspects of the invention, the antibody or antigen-binding fragment thereof comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) identical to the amino acid sequence set forth in SEQ ID NO: 13. In certain aspects of the invention, the antibody or antigen-binding fragment thereof comprises the amino acid sequence set forth in SEQ ID NO: 13 or a conservative amino acid substitution thereof. In certain aspects of the invention, the antibody or antigen-binding fragment thereof comprises the amino acid sequence set forth in SEQ ID NO: 13. In certain aspects of the invention, the antibody or antigen-binding fragment thereof consists of the amino acid sequence set forth in SEQ ID NO: 13. SEQ ID NO: 13 is provided below:

SSGGGGSGGGGSGGGGSASDIQMTQSPSSLSASVGDRVTITCRAS
QDVNTAVAWYQQKPGKAPKLLIYSASFLYSGVPSRFSGSRSGTDF
NLTISSLQPEDFATYYCQQHYTTPPTFGQGTKVEIKGGGGSGGGG
SGGGGSEVQLVESGGGLVQPGGSLRLSCAASGFNIKDTYIHWVRQ
APGKGLEWVARIYLTNGYTRYADSVKGRFTISADTSKNTTYLQMN
SLRAEDTAVYYCSRWGGDGFYAMDYWGQGTLVTVSSRAAIDAKTT
APSVYPLALVSSGSGSL

SEQ ID NO: 13 includes a first linker at the beginning and second linker at the end of the SCA. SEQ ID NO: 13 includes a mutation in the second linker, P278L, which is indicated in bold in the sequence above (the bold L). The T91N mutation is also indicated as a bold N.

In certain aspects of the invention, the antibody or antigen-binding fragment thereof set forth in SEQ ID NO: 13 is encoded by a nucleotide sequence set forth in SEQ ID NO: 14, which is provided below:

agctcaggtggaggcggttcaggcggaggtggctctggcggtggc
ggatctgctagcgatatccagatgacccagtccccgagctccctg
tccgcctctgtgggcgatagggtcaccatcacctgccgtgccagt
caggatgtgaatactgctgtagcctggtatcaacagaaaccagga
aaagctccgaaactactgatttactcggcatccttcctctactct
ggagtcccttctcgcttctctggatccagatctgggacggatttc
aAtctgaccatcagcagtctgcagccggaagacttcgcaacttat
tactgtcagcaacattatactactcctcccacgttcggacagggt
accaaggtggagatcaaaggtggaggtggatcaggtggaggtgga
tcaggtggaggtggatcagaggttcagctggtggagtctggcggt
ggcctggtgcagccagggggctcactccgtttgtcctgtgcagct
tctggcttcaacattaaagacacctatatacactgggtgcgtcag
gccccgggtaagggcctggaatgggttgcaaggatttatcttacg
aatggttatactagatatgccgatagcgtcaagggccgtttcact
ataagcgcagacacatccaaaaacacaacctacctgcagatgaac
agtctgcgtgctgaggacactgccgtctattattgttctagatgg
ggaggggacggcttctatgctatggactactggggtcaaggaacc
ctggtcacagtctcctcgcgtgcggccatcgatgctaaaacgacg
gcaccgtctgtctacccactggcacttgtctcgtctggatccggg
tctctg

In certain aspects of the invention, the antibody or antigen-binding fragment thereof comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous to the amino acid sequence set forth in SEQ ID NO: 15. In certain aspects of the invention, the antibody or antigen-binding fragment thereof comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) identical to the amino acid sequence set forth in SEQ ID NO: 15. In certain aspects of the invention, the antibody or antigen-binding fragment thereof comprises the amino acid sequence set forth in SEQ ID NO: 15 or a conservative amino acid substitution thereof. In certain aspects of the invention, the antibody or antigen-binding fragment thereof comprises the amino acid sequence set forth in SEQ ID NO: 15. In certain aspects of the invention, the antibody or antigen-binding fragment thereof consists of the amino acid sequence set forth in SEQ ID NO: 15. SEQ ID NO: 15 is provided below:

SSGGGGSGGGGSGGGGSASDIQMTQSPSSLSASVGDRVTITCRAS
QDVNTAVAWYQQKPGKAPKLLIYSASFLYSGVPSRFSGSRSGTDF
TLNISSLQPEDFATYYCQQHYTTPPTFGQGTKVEIKGGGGSGGGG
SGGGGSEVQLVESGGGLVQPGGSLRLSCAASGFNIKDTYIHWVRQ
APGKGLEWVARIYLTNGYTRYADSVKGRFTISADTSKNTTYLQMN
SLRAEDTAVYYCSRWGGDGFYAMDYWGQGTLVTVSSRAAIDAKTT
APSVYPLALVSSGSGSL

SEQ ID NO: 15 includes a first linker at the beginning and second linker at the end of the SCA. SEQ ID NO: 15 includes a mutation in the second linker, P278L, which is indicated in bold in the sequence above (the bold L). The T91N mutation is also indicated as a bold N.

In certain aspects of the invention, the antibody or antigen-binding fragment thereof set forth in SEQ ID NO: 15 is encoded by a nucleotide sequence set forth in SEQ ID NO: 16, which is provided below:

agctcaggtggaggcggttcaggcggaggtggctctggcggtggc
ggatctgctagcgatatccagatgacccagtccccgagctccctg
tccgcctctgtgggcgatagggtcaccatcacctgccgtgccagt
caggatgtgaatactgctgtagcctggtatcaacagaaaccagga
aaagctccgaaactactgatttactcggcatccttcctctactct
ggagtcccttctcgcttctctggatccagatctgggacggatttc
actctgaAcatcagcagtctgcagccggaagacttcgcaacttat
tactgtcagcaacattatactactcctcccacgttcggacagggt
accaaggtggagatcaaaggtggaggtggatcaggtggaggtgga
tcaggtggaggtggatcagaggttcagctggtggagtctggcggt
ggcctggtgcagccagggggctcactccgtttgtcctgtgcagct
tctggcttcaacattaaagacacctatatacactgggtgcgtcag
gccccgggtaagggcctggaatgggttgcaaggatttatcttacg
aatggttatactagatatgccgatagcgtcaagggccgtttcact
ataagcgcagacacatccaaaaacacaacctacctgcagatgaac
agtctgcgtgctgaggacactgccgtctattattgttctagatgg
ggaggggacggcttctatgctatggactactggggtcaaggaacc
ctggtcacagtctcctcgcgtgcggccatcgatgctaaaacgacg
gcaccgtctgtctacccactggcacttgtctcgtctggatccggg
tctctg

In certain aspects of the invention, the antibody or antigen-binding fragment thereof comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous to the amino acid sequence set forth in SEQ ID NO: 17. In certain aspects of the invention, the antibody or antigen-binding fragment thereof comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) identical to the amino acid sequence set forth in SEQ ID NO: 17. In certain aspects of the invention, the antibody or antigen-binding fragment thereof comprises the amino acid sequence set forth in SEQ ID NO: 17 or a conservative amino acid substitution thereof. In certain aspects of the invention, the antibody or antigen-binding fragment thereof comprises the amino acid sequence set forth in SEQ ID NO: 17. In certain aspects of the invention, the antibody or antigen-binding fragment thereof consists of the amino acid sequence set forth in SEQ ID NO: 17. SEQ ID NO: 17 is provided below:

SSGGGGSGGGGSGGGGSASDIQMTQSPSSLSASVGDRVTITCRAS
QDVNTAVAWYQQKPGKAPKLLIYSASFLYSGVPSRFSGSRSGTDF
NLNISSLOPEDFATYYCQQHYTTPPTFGQGTKVEIKGGGGSGGGG
SGGGGSEVQLVESGGGLVQPGGSLRLSCAASGFNIKDTYIHWVRQ
APGKGLEWVARIYLTNGYTRYADSVKGRFTISADTSKNTTYLQMN
SLRAEDTAVYYCSRWGGDGFYAMDYWGQGTLVTVSSRAAIDAKTT
APSVYPLALVSSGSGSL

SEQ ID NO: 17 includes a first linker at the beginning and second linker at the end of the SCA. SEQ ID NO: 17 includes a mutation in the second linker, P278L, which is indicated in bold in the sequence above (the bold L). The T91N mutation is also indicated as a bold N.

In certain aspects of the invention, the antibody or antigen-binding fragment thereof set forth in SEQ ID NO: 17 is encoded by a nucleotide sequence set forth in SEQ ID NO: 18, which is provided below:

agctcaggtggaggcggttcaggcggaggtggctctggcggtggc
ggatctgctagcgatatccagatgacccagtccccgagctccctg
tccgcctctgtgggcgatagggtcaccatcacctgccgtgccagt
caggatgtgaatactgctgtagcctggtatcaacagaaaccagga
aaagctccgaaactactgatttactcggcatccttcctctactct
ggagtcccttctcgcttctctggatccagatctgggacggatttc
aAtctgaAcatcagcagtctgcagccggaagacttcgcaacttat
tactgtcagcaacattatactactcctcccacgttcggacagggt
accaaggtggagatcaaaggtggaggtggatcaggtggaggtgga
tcaggtggaggtggatcagaggttcagctggtggagtctggcggt
ggcctggtgcagccagggggctcactccgtttgtcctgtgcagct
tctggcttcaacattaaagacacctatatacactgggtgcgtcag
gccccgggtaagggcctggaatgggttgcaaggatttatcttacg
aatggttatactagatatgccgatagcgtcaagggccgtttcact
ataagcgcagacacatccaaaaacacaacctacctgcagatgaac
agtctgcgtgctgaggacactgccgtctattattgttctagatgg
ggaggggacggcttctatgctatggactactggggtcaaggaacc
ctggtcacagtctcctcgcgtgcggccatcgatgctaaaacgacg
gcaccgtctgtctacccactggcacttgtctcgtctggatccggg
tctctg

In certain aspects of the invention, conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. Amino acids can also be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. In certain aspects of the invention, no more than one, no more than two, no more than three, no more than four, no more than five residues within a specified sequence are altered. Exemplary conservative amino acid substitutions are shown in Table 1 below.

TABLE 1

| Original Residue | Exemplary Conservative Amino Acid Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

As used herein, the percent homology between two amino acid sequences is equivalent to the percent identity between the two sequences. The percent identity between the two sequences is a function of the number of identical positions shared by the sequences (i.e., % homology=# of identical positions/total # of positions ×100), considering the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4: 11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol., 48: 444-453 (1970)) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

The nucleic acid molecule encoding the anti-HER2 antibody or antigen-binding fragment thereof can be a DNA molecule, an RNA molecule, or a cDNA molecule to conform to the nucleic acid of the viral genome into which it is integrated.

In certain aspects of the invention, the surface protein associated with infectivity includes a glycoprotein or a modified glycoprotein. Glycoprotein g has been attributed to the binding and fusion mechanisms of the vesicular stomatitis virus during infection. In certain aspects of the invention, the glycoprotein is a vesicular stomatitis virus glycoprotein, VSV-G. Physiologically, VSV-G exhibits a remarkably robust and pantropic infectivity, mediated by its coat protein. The LDL receptor (LDLR) serves as the major entry port of VSV and through the VSV-G binding (see also Proc. Natl. Acad. Sci. U.S.A. 110: 7306-7311(2013)). In certain aspects of the invention, the glycoprotein is modified to exhibit reduced binding and/or infectivity. In certain aspects of the invention, binding is reduced by at least about 50%, at least about 75%, at least about 80%, at least about 85%, at least about 90%, at least about 95%, at least about 96%, at least about 97%, at least about 98%, at least about 99% or more when compared to a non-modified glycoprotein (e.g., naturally occurring). In certain aspects of the invention, infectivity is reduced by at least about 50%, at least about 75%, at least about 80%, at least about 85%, at least about 90%, at least about 95%, at least about 96%, at least about 97%, at least about 98%, at least about 99% or more when compared to a non-modified glycoprotein (e.g., naturally occurring). In certain aspects of the invention, the modification can be an insertion, a deletion, or a substitution of one or more amino acids. In certain aspects of the invention, the modification can be a substitution of one or more amino acids.

In certain aspects of the invention, the glycoprotein comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous to the amino acid sequence set forth in SEQ ID NO: 19. In certain aspects of the invention, the glycoprotein comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) identical to the amino acid sequence set forth in SEQ ID NO: 19. In certain aspects of the invention, the glycoprotein comprises the amino acid sequence set forth in SEQ ID NO: 19. In certain aspects of the invention, the glycoprotein consists of the amino acid sequence set forth in SEQ ID NO: 19. SEQ ID NO: 19 is provided below:

MKCLLYLAFLFIGVNCKFTIVFPHNQKGNWKNVPSNYHYCPSSSD
LNWHNDLIGTALQVKMPKSHKAIQADGWMCHASKWVTTCDFRWYG
PKYITHSIRSFTPSVEQCKESIEQTKQGTWLNPGFPPQSCGYATV
IDAEAVIVQVTPHHVLVDEYTGEWVDSQFINGKCSNYICPTVHNS
TTWHSDYKVKGLCDSNLISMDITFFSEDGELSSLGKEGTGFRSNY
FAYETGGKACKMQYCKHWGVRLPSGVWFEMADKDLFAAARFPECP
EGSSISAPSQTSVDVSLIQDVERILDYSLCQETWSKIRAGLPISP
VDLSYLAPKNPGTGPAFTIINGTLKYFETRYIRVDIAAPILSRMV
GMISGTTTERELWDDWAPYEDVEIGPNGVLRTSSGYKFPLYMIGH
GMLDSDLHLSSKAQVFEHPHIQDAASQLPDDESLFFGDTGLSKNP
IELVEGWFSSWKSSIASFFFIIGLIIGLFLVLRVGIHLCIKLKHT
KKRQIYTDIEMNRLGK*

In certain aspects, the glycoprotein set forth in SEQ ID NO: 19 is encoded by a nucleotide sequence set forth in SEQ ID NO: 20, which is provided below:

ATGAAGTGCCTTTTGTACTTAGCCTTTTTATTCATTGGGGTGAAT
TGCAAGTTCACCATAGTTTTTCCACACAACCAAAAAGGAAACTGG
AAAAATGTTCCTTCTAATTACCATTATTGCCCGTCAAGCTCAGAT
TTAAATTGGCATAATGACTTAATAGGCACAGCCTTACAAGTCAAA
ATGCCCAAGAGTCACAAGGCTATTCAAGCAGACGGTTGGATGTGT
CATGCTTCCAAATGGGTCACTACTTGTGATTTCCGCTGGTATGGA
CCGAAGTATATAACACATTCCATCCGATCCTTCACTCCATCTGTA
GAACAATGCAAGGAAAGCATTGAACAAACGAAACAAGGAACTTGG
CTGAATCCAGGCTTCCCTCCTCAAAGTIGTGGATATGCAACTGTG
ACGGATGCCGAAGCAGTGATTGTCCAGGTGACTCCTCACCATGTG
CTGGTTGATGAATACACAGGAGAATGGGTTGATTCACAGTTCATC
AACGGAAAATGCAGCAATTACATATGCCCCACTGTCCATAACTCT
ACAACCTGGCATTCTGACTATAAGGTCAAAGGGCTATGTGATTCT
AACCTCATTTCCATGGACATCACCTTCTTCTCAGAGGACGGAGAG
CTATCATCCCTGGGAAAGGAGGGCACAGGGTTCAGAAGTAACTAC
TTTGCTTATGAAACTGGAGGCAAGGCCTGCAAAATGCAATACTGC
AAGCATTGGGGAGTCAGACTCCCATCAGGTGTCTGGTTCGAGATG
GCTGATAAGGATCTCTTTGCTGCAGCCAGATTCCCTGAATGCCCA
GAAGGGTCAAGTATCTCTGCTCCATCTCAGACCTCAGTGGATGTA
AGTCTAATTCAGGACGTTGAGAGGATCTTGGATTATTCCCTCTGC
CAAGAAACCTGGAGCAAAATCAGAGCGGGTCTTCCAATCTCTCCA
GTGGATCTCAGCTATCTTGCTCCTAAAAACCCAGGAACCGGTCCT
GCTTTCACCATAATCAATGGTACCCTAAAATACTTTGAGACCAGA
TACATCAGAGTCGATATTGCTGCTCCAATCCTCTCAAGAATGGTC
GGAATGATCAGTGGAACTACCACAGAAAGGGAACTGTGGGATGAC
TGGGCACCATATGAAGACGTGGAAATTGGACCCAATGGAGTTCTG
AGGACCAGTTCAGGATATAAGTTTCCTTTATACATGATTGGACAT
GGTATGTTGGACTCCGATCTTCATCTTAGCTCAAAGGCTCAGGTG
TTCGAACATCCTCACATTCAAGACGCTGCTTCGCAACTTCCTGAT
GATGAGAGTTTATTTTTTGGTGATACTGGGCTATCCAAAAATCCA
ATCGAGCTTGTAGAAGGTTGGTTCAGTAGTTGGAAAAGCTCTATT
GCCTCTTTTTTCTTTATCATAGGGTTAATCATTGGACTATTCTTG
GTTCTCCGAGTTGGTATCCATCTTTGCATTAAATTAAAGCACACC
AAGAAAAGACAGATTTATACAGACATAGAGATGAACCGACTTGGA
AAGTAA

In certain aspects of the invention, the glycoprotein comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous to the amino acid sequence set forth in SEQ ID NO: 21. In certain aspects of the invention, the glycoprotein comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) identical to the amino acid sequence set forth in SEQ ID NO: 21. In certain aspects of the invention, the glycoprotein comprises the amino acid sequence set forth in SEQ ID NO: 21. In certain aspects of the invention, the glycoprotein consists of the amino acid sequence set forth in SEQ ID NO: 21. SEQ ID NO: 21 is provided below:

KFTIVFPHNQKGNWKNVPSNYHYCPSSSDLNWHNDLIGTALQVKM
PKSHKAIQADGWMCHASKWVTTCDFRWYGPKYITHSIRSFTPSVE
QCKESIEQTKQGTWLNPGFPPQSCGYATVTDAEAVIVQVTPHHVL
VDEYTGEWVDSQFINGKCSNYICPTVHNSTTWHSDYKVKGLCDSN
LISMDITFFSEDGELSSLGKEGTGFRSNYFAYETGGKACKMQYCK
HWGVRLPSGVWFEMADKDLFAAARFPECPEGSSISAPSQTSVDVS
LIQDVERILDYSLCQETWSKIRAGLPISPVDLSYLAPKNPGTGPA
FTIINGTLKYFETRYIRVDIAAPILSRMVGMISGTTTERELWDDW
APYEDVEIGPNGVLRTSSGYKFPLYMIGHGMLDSDLHLSSKAQVF
EHPHIQDAASQLPDDESLFFGDTGLSKNPIELVEGWFSSWKSSIA
SFFFIIGLIIGLFLVLRVGIHLCIKLKHTKKRQIYTDIEMNRLGK*

In certain aspects of the invention, the glycoprotein set forth in SEQ ID NO: 21 is encoded by a nucleotide sequence set forth in SEQ ID NO: 22, which is provided below:

AAGTTCACCATAGTTTTTCCACACAACCAAAAAGGAAACTGGAAA
AATGTTCCTTCTAATTACCATTATTGCCCGTCAAGCTCAGATTTA
AATTGGCATAATGACTTAATAGGCACAGCCTTACAAGTCAAAATG
CCCAAGAGTCACAAGGCTATTCAAGCAGACGGTTGGATGTGTCAT
GCTTCCAAATGGGTCACTACTTGTGATTTCCGCTGGTATGGACCG
AAGTATATAACACATTCCATCCGATCCTTCACTCCATCTGTAGAA
CAATGCAAGGAAAGCATTGAACAAACGAAACAAGGAACTTGGCTG
AATCCAGGCTTCCCTCCTCAAAGTTGTGGATATGCAACTGTGACG
GATGCCGAAGCAGTGATTGTCCAGGTGACTCCTCACCATGTGCTG
GTTGATGAATACACAGGAGAATGGGTTGATTCACAGTTCATCAAC
GGAAAATGCAGCAATTACATATGCCCCACTGTCCATAACTCTACA
ACCTGGCATTCTGACTATAAGGTCAAAGGGCTATGTGATTCTAAC
CTCATTTCCATGGACATCACCTTCTTCTCAGAGGACGGAGAGCTA
TCATCCCTGGGAAAGGAGGGCACAGGGTTCAGAAGTAACTACTTT
GCTTATGAAACTGGAGGCAAGGCCTGCAAAATGCAATACTGCAAG
CATTGGGGAGTCAGACTCCCATCAGGTGTCTGGTTCGAGATGGCT
GATAAGGATCTCTTTGCTGCAGCCAGATTCCCTGAATGCCCAGAA
GGGTCAAGTATCTCTGCTCCATCTCAGACCTCAGTGGATGTAAGT
CTAATTCAGGACGTTGAGAGGATCTTGGATTATTCCCTCTGCCAA
GAAACCTGGAGCAAAATCAGAGCGGGTCTTCCAATCTCTCCAGTG
GATCTCAGCTATCTTGCTCCTAAAAACCCAGGAACCGGTCCTGCT
TTCACCATAATCAATGGTACCCTAAAATACTTTGAGACCAGATAC
ATCAGAGTCGATATTGCTGCTCCAATCCTCTCAAGAATGGTCGGA
ATGATCAGTGGAACTACCACAGAAAGGGAACTGTGGGATGACTGG
GCACCATATGAAGACGTGGAAATTGGACCCAATGGAGTTCTGAGG
ACCAGTTCAGGATATAAGTTTCCTTTATACATGATTGGACATGGT
ATGTTGGACTCCGATCTTCATCTTAGCTCAAAGGCTCAGGTGTTC
GAACATCCTCACATTCAAGACGCTGCTTCGCAACTTCCTGATGAT
GAGAGTTTATTTTTTGGTGATACTGGGCTATCCAAAAATCCAATC
GAGCTTGTAGAAGGTTGGTTCAGTAGTTGGAAAAGCTCTATTGCC
TCTTTTTTCTTTATCATAGGGTTAATCATTGGACTATTCTTGGTT
CTCCGAGTTGGTATCCATCTTTGCATTAAATTAAAGCACACCAAG
AAAAGACAGATTTATACAGACATAGAGATGAACCGACTTGGAAAG
TAA

In certain aspects of the invention, the glycoprotein can be modified by insertion, deletion, or substitution of one or more amino acid residues. In certain non-limiting aspects, the glycoprotein can be modified by insertion, deletion, or substitution of one or more amino acid residues at position 10, 47, 156, 184, 194, 238, 354, and 397 of SEQ ID NO: 21. In certain non-limiting aspects, the glycoprotein can include a substitution at position 10 of SEQ ID NO: 21, a substitution at position 47 of SEQ ID NO: 21, a substitution at position 156 of SEQ ID NO: 21, a substitution at position 184 of SEQ ID NO: 21, a substitution at position 194 of SEQ ID NO: 21, a substitution at position 238 of SEQ ID NO: 21, a substitution at position 354 of SEQ ID NO: 21, a substitution at position 397 of SEQ ID NO: 21, or a combination thereof. In certain non-limiting aspects, the glycoprotein can include a substitution of a glutamine residue with a lysine residue at position 10 of SEQ ID NO: 21, a substitution of a lysine residue with a glutamine residue at position 47 of SEQ ID NO: 21, a substitution of a tyrosine residue with an aspartic acid residue at position 156 of SEQ ID NO: 21, a substitution of a methionine residue with a threonine residue at position 184 of SEQ ID NO: 21, a substitution of a glutamic acid residue with a lysine residue at position 194 of SEQ ID NO: 21, a substitution of a glutamic acid residue with a lysine residue at position 238 of SEQ ID NO: 21, a substitution of an arginine residue with a glutamine residue at position 354 of SEQ ID NO: 21, a substitution of an histidine residue with an arginine residue at position 397 of SEQ ID NO: 21, or a combination thereof. Standard techniques, including genetic engineering methods and site directed mutagenesis may be performed to create specific mutations.

In certain aspects of the invention, the glycoprotein includes a substitution of a glutamine residue with a lysine residue at position 10 of SEQ ID NO: 21. In certain aspects, the glycoprotein includes a substitution of a lysine residue with a glutamine residue at position 47 of SEQ ID NO: 21. In certain aspects of the invention, the glycoprotein includes a substitution of a glutamine residue with a lysine residue at position 10 of SEQ ID NO: 21 and a substitution of a lysine residue with a glutamine residue at position 47 of SEQ ID NO: 21. In certain aspects, the glycoprotein can include a substitution of a tyrosine residue with an aspartic acid residue at position 156 of SEQ ID NO: 21. In certain aspects, the glycoprotein can include a substitution of a methionine residue with a threonine residue at position 184 of SEQ ID NO: 21. In certain aspects, the glycoprotein can include a substitution of a glutamic acid residue with a lysine residue at position 194 of SEQ ID NO: 21. In certain aspects, the glycoprotein can include a substitution of a glutamic acid residue with a lysine residue at position 238 of SEQ ID NO: 21. In certain aspects, the glycoprotein can include a substitution of an arginine residue with a glutamine residue at position 354 of SEQ ID NO: 21. In certain aspects, the glycoprotein can include a substitution of an histidine residue with an arginine residue at position 397 of SEQ ID NO: 21.

In certain aspects of the invention, the glycoprotein comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous to the amino acid sequence set forth in SEQ ID NO: 23. In certain aspects of the invention, the glycoprotein comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) identical to the amino acid sequence set forth in SEQ ID NO: 23. In certain aspects of the invention, the glycoprotein comprises the amino acid sequence set forth in SEQ ID NO: 23. In certain aspects of the invention, the glycoprotein consists of the amino acid sequence set forth in SEQ ID NO: 23. SEQ ID NO: 23 is provided below:

MKCLLYLAFLFIGVNCKFTIVFPHNQKGNWKNVPSNYHYCPSSSD
LNWHNDLIGTALQVKMPQSHKAIQADGWMCHASKWVTTCDFRWYG
PKYITHSIRSFTPSVEQCKESIEQTKQGTWLNPGFPPQSCGYATV
IDAEAVIVQVTPHHVLVDEYTGEWVDSQFINGKCSNYICPTVHNS
TTWHSDYKVKGLCDSNLISMDITFFSEDGELSSLGKEGTGFRSNY
FAYETGGKACKMQYCKHWGVRLPSGVWFEMADKDLFAAARFPECP
EGSSISAPSQTSVDVSLIQDVERILDYSLCQETWSKIRAGLPISP
VDLSYLAPKNPGTGPAFTIINGTLKYFETRYIRVDIAAPILSRMV
GMISGTTTEQELWDDWAPYEDVEIGPNGVLRTSSGYKFPLYMIGH
GMLDSDLHLSSKAQVFEHPHIQDAASQLPDDESLFFGDTGLSKNP
IELVEGWFSSWKSSIASFFFIIGLIIGLFLVLRVGIHLCIKLKHT
KKRQIYTDIEMNRLGK*

In certain aspects of the invention, the glycoprotein set forth in SEQ ID NO: 23 is encoded by a nucleotide sequence set forth in SEQ ID NO: 24, which is provided below:

ATGAAGTGCCTTTTGTACTTAGCCTTTTTATTCATTGGGGTGAAT
TGCAAGTTCACCATAGTTTTTCCACACAACCAAAAAGGAAACTGG
AAAAATGTTCCTTCTAATTACCATTATTGCCCGTCAAGCTCAGAT
TTAAATTGGCATAATGACTTAATAGGCACAGCCTTACAAGTCAAA
ATGCCCCAGAGTCACAAGGCTATTCAAGCAGACGGTTGGATGTGT
CATGCTTCCAAATGGGTCACTACTTGTGATTTCCGCTGGTATGGA
CCGAAGTATATAACACATTCCATCCGATCCTTCACTCCATCTGTA
GAACAATGCAAGGAAAGCATTGAACAAACGAAACAAGGAACTTGG
CTGAATCCAGGCTTCCCTCCTCAAAGTTGTGGATATGCAACTGTG
ACGGATGCCGAAGCAGTGATTGTCCAGGTGACTCCTCACCATGTG
CTGGTTGATGAATACACAGGAGAATGGGTTGATTCACAGTTCATC
AACGGAAAATGCAGCAATTACATATGCCCCACTGTCCATAACTCT
ACAACCTGGCATTCTGACTATAAGGTCAAAGGGCTATGTGATTCT
AACCTCATTTCCATGGACATCACCTTCTTCTCAGAGGACGGAGAG
CTATCATCCCTGGGAAAGGAGGGCACAGGGTTCAGAAGTAACTAC
TTTGCTTATGAAACTGGAGGCAAGGCCTGCAAAATGCAATACTGC
AAGCATTGGGGAGTCAGACTCCCATCAGGTGTCTGGTTCGAGATG
GCTGATAAGGATCTCTTTGCTGCAGCCAGATTCCCTGAATGCCCA
GAAGGGTCAAGTATCTCTGCTCCATCTCAGACCTCAGTGGATGTA
AGTCTAATTCAGGACGTTGAGAGGATCTTGGATTATTCCCTCTGC
CAAGAAACCTGGAGCAAAATCAGAGCGGGTCTTCCAATCTCTCCA
GTGGATCTCAGCTATCTTGCTCCTAAAAACCCAGGAACCGGTCCT
GCTTTCACCATAATCAATGGTACCCTAAAATACTTTGAGACCAGA
TACATCAGAGTCGATATTGCTGCTCCAATCCTCTCAAGAATGGTC
GGAATGATCAGTGGAACTACCACAGAACAGGAACTGTGGGATGAC
TGGGCACCATATGAAGACGTGGAAATTGGACCCAATGGAGTTCTG
AGGACCAGTTCAGGATATAAGTTTCCTTTATACATGATTGGACAT
GGTATGTTGGACTCCGATCTTCATCTTAGCTCAAAGGCTCAGGTG
TTCGAACATCCTCACATTCAAGACGCTGCTTCGCAACTTCCTGAT
GATGAGAGTTTATTTTTTGGTGATACTGGGCTATCCAAAAATCCA
ATCGAGCTTGTAGAAGGTTGGTTCAGTAGTTGGAAAAGCTCTATT
GCCTCTTTTTTCTTTATCATAGGGTTAATCATTGGACTATTCTTG
GTTCTCCGAGTTGGTATCCATCTTTGCATTAAATTAAAGCACACC
AAGAAAAGACAGATTTATACAGACATAGAGATGAACCGACTTGGA
AAGTA

In certain aspects of the invention, the glycoprotein comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous to the amino acid sequence set forth in SEQ ID NO: 25. In certain aspects of the invention, the glycoprotein comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) identical to the amino acid sequence set forth in SEQ ID NO: 25. In certain aspects of the invention, the glycoprotein comprises the amino acid sequence set forth in SEQ ID NO: 25. In certain aspects of the invention, the glycoprotein consists of the amino acid sequence set forth in SEQ ID NO: 25. SEQ ID NO: 25 is provided below:

KFTIVFPHNKKGNWKNVPSNYHYCPSSSDLNWHNDLIGTALQVKM
PQSHKAIQADGWMCHASKWVTTCDFRWYGPKYITHSIRSFTPSVE
QCKESIEQTKQGTWLNPGFPPQSCGYATVTDAEAVIVQVTPHHVL
VDEYTGEWVDSQFINGKCSNDICPTVHNSTTWHSDYKVKGLCDSN
LISTDITFFSEDGKLSSLGKEGTGFRSNYFAYETGGKACKMQYCK
HWGVRLPSGVWFKMADKDLFAAARFPECPEGSSISAPSQTSVDVS
LIQDVERILDYSLCQETWSKIRAGLPISPVDLSYLAPKNPGTGPA
FTIINGTLKYFETRYIRVDIAAPILSRMVGMISGTTTEQELWDDW
APYEDVEIGPNGVLRTSSGYKFPLYMIGHGMLDSDLRLSSKAQVF
EHPHIQDAASQLPDDESLFFGDTGLSKNPIELVEGWFSSWKSSIA
SFFFIIGLIIGLFLVLRVGIHLCIKLKHTKKRQIYTDIEMNRLGK*

In certain aspects of the invention, the glycoprotein set forth in SEQ ID NO: 25 is encoded by a nucleotide sequence set forth in SEQ ID NO: 26, which is provided below:

AAGTTCACCATAGTTTTTCCACACAACAAGAAAGGAAACTGGAAA
AATGTTCCTTCTAATTACCATTATTGCCCGTCAAGCTCAGATTTA
AATTGGCATAATGACTTAATAGGCACAGCCTTACAAGTCAAAATG
CCCCAGAGTCACAAGGCTATTCAAGCAGACGGTTGGATGTGTCAT
GCTTCCAAATGGGTCACTACTTGTGATTTCCGCTGGTATGGACCG
AAGTATATAACACATTCCATCCGATCCTTCACTCCATCTGTAGAA
CAATGCAAGGAAAGCATTGAACAAACGAAACAAGGAACTTGGCTG
AATCCAGGCTTCCCTCCTCAAAGTTGTGGATATGCAACTGTGACG
GATGCCGAAGCAGTGATTGTCCAGGTGACTCCTCACCATGTGCTG
GTTGATGAATACACAGGAGAATGGGTTGATTCACAGTTCATCAAC
GGAAAATGCAGCAATGACATATGCCCCACTGTCCATAACTCTACA
ACCTGGCATTCTGACTATAAGGTCAAAGGGCTATGTGATTCTAAC
CTCATTTCCACGGACATCACCTTCTTCTCAGAGGACGGAAAGCTA
TCATCCCTGGGAAAGGAGGGCACAGGGTTCAGAAGTAACTACTTT
GCTTATGAAACTGGAGGCAAGGCCTGCAAAATGCAATACTGCAAG
CATTGGGGAGTCAGACTCCCATCAGGTGTCTGGTTCAAGATGGCT
GATAAGGATCTCTTTGCTGCAGCCAGATTCCCTGAATGCCCAGAA
GGGTCAAGTATCTCTGCTCCATCTCAGACCTCAGTGGATGTAAGT
CTAATTCAGGACGTTGAGAGGATCTTGGATTATTCCCTCTGCCAA
GAAACCTGGAGCAAAATCAGAGCGGGTCTTCCAATCTCTCCAGTG
GATCTCAGCTATCTTGCTCCTAAAAACCCAGGAACCGGTCCTGCT
TTCACCATAATCAATGGTACCCTAAAATACTTTGAGACCAGATAC
ATCAGAGTCGATATTGCTGCTCCAATCCTCTCAAGAATGGTCGGA
ATGATCAGTGGAACTACCACAGAACAGGAACTGTGGGATGACTGG
GCACCATATGAAGACGTGGAAATTGGACCCAATGGAGTTCTGAGG
ACCAGTTCAGGATATAAGTTTCCTTTATACATGATTGGACATGGT
ATGTTGGACTCCGATCTTCGTCTTAGCTCAAAGGCTCAGGTGTTC
GAACATCCTCACATTCAAGACGCTGCTTCGCAACTTCCTGATGAT
GAGAGTTTATTTTTTGGTGATACTGGGCTATCCAAAAATCCAATC
GAGCTTGTAGAAGGTTGGTTCAGTAGTTGGAAAAGCTCTATTGCC
TCTTTTTTCTTTATCATAGGGTTAATCATTGGACTATTCTTGGTT
CTCCGAGTTGGTATCCATCTTTGCATTAAATTAAAGCACACCAAG
AAAAGACAGATTTATACAGACATAGAGATGAACCGACTTGGAAAG
TAA

In certain aspects of the invention, the glycoprotein consists of a chimeric protein that has the following elements: signal sequence-first linker-SCA-second linker-G.

In aspects of the invention, the first linker is a “Glycine-Serine” linker. In an aspect, the Glycine-Serine linker may contain various quantities of amino acid residues. In some aspects the Glycine-Serine linker comprises 1, 2, 3, 4, 5, 6, 7, 8, 9, or 10 G⁴S or G⁴A repeats.

In aspects of the invention, the first linker comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous to the amino acid sequence set forth in SEQ ID NO: 29. In certain aspects of the invention, the first linker comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) identical to the amino acid sequence set forth in SEQ ID NO: 29. In certain aspects of the invention, the first linker comprises the amino acid sequence set forth in SEQ ID NO: 29. In certain aspects of the invention, the first linker consists of the amino acid sequence set forth in SEQ ID NO: 29. SEQ ID NO: 29 is provided below:

SSGGGGSGGGGSGGGGSAS

In certain aspects of the invention, the first linker consists of the nucleotide sequence set forth in SEQ ID NO: 30:

agctcaggtggaggcggttcaggcggaggtggctctggcggtggc
ggatctgctagc

In aspects of the invention, the second linker comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous to the amino acid sequence set forth in SEQ ID NO: 31. In certain aspects of the invention, the second linker comprises an amino acid sequence that is at least about 80%, at least about 85%, at least about 90%, at about least 95%, at about least 96%, at least about 97%, at least about 98%, or at least about 99% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) identical to the amino acid sequence set forth in SEQ ID NO: 31. In certain aspects of the invention, the second linker comprises the amino acid sequence set forth in SEQ ID NO: 31. In certain aspects of the invention, the second linker consists of the amino acid sequence set forth in SEQ ID NO: 31. SEQ ID NO: 31 is provided below:

IDAKTTAPSVYPLAPVSSGSGSL

In certain aspects of the invention, the second linker has been adapted and consists of the amino acid sequence set forth in SEQ ID NO: 33:

IDAKTTAPSVYPLALVSSGSGSL

In certain aspects of the invention, the second linker consists of the nucleotide sequence set forth in SEQ ID NO: 32:

atcgatgctaaaacgacggcaccgtctgtctacccactggcacct
gtctcgtctggatccgggtctctg

In certain aspects of the invention, the second linker has been adapted and consists of the nucleotide sequence set forth in SEQ ID NO: 34:

atcgatgctaaaacgacggcaccgtctgtctacccactggcactt
gtctcgtctggatccgggtctctg

In certain aspects of the invention, the chimeric glycoprotein consists of the amino acid sequence set forth in SEQ ID NO: 27:

MKCLLYLAFLFIGVNCSSGGGGSGGGGSGGGGSASDIQMTQSPSS
LSASVGDRVTITCRASQDVNTAVAWYQQKPGKAPKLLIYSASFLY
SGVPSRFSGSRSGTDFNLNISSLQPEDFATYYCQQHYTTPPTFGQ
GTKVEIKGGGGSGGGGSGGGGSEVQLVESGGGLVQPGGSLRLSCA
ASGFNIKDTYIHWVRQAPGKGLEWVARIYLTNGYTRYADSVKGRF
TISADTSKNTTYLQMNSLRAEDTAVYYCSRWGGDGFYAMDYWGQG
TLVTVSSRAAIDAKTTAPSVYPLALVSSGSGSLKFTIVFPHNKKG
NWKNVPSNYHYCPSSSDLNWHNDLIGTALQVKMPQSHKAIQADGW
MCHASKWVTTCDFRWYGPKYITHSIRSFTPSVEQCKESIEQTKQG
TWLNPGFPPQSCGYATVTDAEAVIVQVTPHHVLVDEYTGEWVDSQ
FINGKCSNDICPTVHNSTTWHSDYKVKGLCDSNLISTDITFFSED
GKLSSLGKEGTGFRSNYFAYETGGKACKMQYCKHWGVRLPSGVWF
KMADKDLFAAARFPECPEGSSISAPSQTSVDVSLIQDVERILDYS
LCQETWSKIRAGLPISPVDLSYLAPKNPGTGPAFTIINGTLKYFE
TRYIRVDIAAPILSRMVGMISGTTTEQELWDDWAPYEDVEIGPNG
VLRTSSGYKFPLYMIGHGMLDSDLRLSSKAQVFEHPHIQDAASQL
PDDESLFFGDTGLSKNPIELVEGWFSSWKSSIASFFFIIGLIIGL
FLVLRVGIHLCIKLKHTKKRQIYTDIEMNRLGK*

In certain aspects of the invention, the chimeric glycoprotein consists of the nucleotide sequence set forth in SEQ ID NO: 28:

ATGAAGTGCCTTTTGTACTTAGCCTTTTTATTCATTGGGGTGAAT
TGCagctcaggtggaggcggttcaggcggaggtggctctggcggt
ggcggatctgctagcgatatccagatgacccagtccccgagctcc
ctgtccgcctctgtgggcgatagggtcaccatcacctgccgtgcc
agtcaggatgtgaatactgctgtagcctggtatcaacagaaacca
ggaaaagctccgaaactactgatttactcggcatccttcctctac
tctggagtcccttctcgcttctctggatccagatctgggacggat
ttcaatctgaacatcagcagtctgcagccggaagacttcgcaact
tattactgtcagcaacattatactactcctcccacgttcggacag
ggtaccaaggtggagatcaaaggtggaggtggatcaggtggaggt
ggatcaggtggaggtggatcagaggttcagctggtggagtctggc
ggtggcctggtgcagccagggggctcactccgtttgtcctgtgca
gcttctggcttcaacattaaagacacctatatacactgggtgcgt
caggccccgggtaagggcctggaatgggttgcaaggatttatctt
acgaatggttatactagatatgccgatagcgtcaagggccgtttc
actataagcgcagacacatccaaaaacacaacctacctgcagatg
aacagtctgcgtgctgaggacactgccgtctattattgttctaga
tggggaggggacggcttctatgctatggactactggggtcaagga
accctggtcacagtctcctcgcgtgcggccatcgatgctaaaacg
acggcaccgtctgtctacccactggcacttgtctcgtctggatcc
gggtctctgAAGTTCACCATAGTTTTTCCACACAACAAGAAAGGA
AACTGGAAAAATGTTCCTTCTAATTACCATTATTGCCCGTCAAGC
TCAGATTTAAATTGGCATAATGACTTAATAGGCACAGCCTTACAA
GTCAAAATGCCCCAGAGTCACAAGGCTATTCAAGCAGACGGTTGG
ATGTGTCATGCTTCCAAATGGGTCACTACTTGTGATTTCCGCTGG
TATGGACCGAAGTATATAACACATTCCATCCGATCCTTCACTCCA
TCTGTAGAACAATGCAAGGAAAGCATTGAACAAACGAAACAAGGA
ACTTGGCTGAATCCAGGCTTCCCTCCTCAAAGTTGTGGATATGCA
ACTGTGACGGATGCCGAAGCAGTGATTGTCCAGGTGACTCCTCAC
CATGTGCTGGTTGATGAATACACAGGAGAATGGGTTGATTCACAG
TTCATCAACGGAAAATGCAGCAATGACATATGCCCCACTGTCCAT
AACTCTACAACCTGGCATTCTGACTATAAGGTCAAAGGGCTATGT
GATTCTAACCTCATTTCCACGGACATCACCTTCTTCTCAGAGGAC
GGAAAGCTATCATCCCTGGGAAAGGAGGGCACAGGGTTCAGAAGT
AACTACTTTGCTTATGAAACTGGAGGCAAGGCCTGCAAAATGCAA
TACTGCAAGCATTGGGGAGTCAGACTCCCATCAGGTGTCTGGTTC
AAGATGGCTGATAAGGATCTCTTTGCTGCAGCCAGATTCCCTGAA
TGCCCAGAAGGGTCAAGTATCTCTGCTCCATCTCAGACCTCAGTG
GATGTAAGTCTAATTCAGGACGTTGAGAGGATCTTGGATTATTCC
CTCTGCCAAGAAACCTGGAGCAAAATCAGAGCGGGTCTTCCAATC
TCTCCAGTGGATCTCAGCTATCTTGCTCCTAAAAACCCAGGAACC
GGTCCTGCTTTCACCATAATCAATGGTACCCTAAAATACTTTGAG
ACCAGATACATCAGAGTCGATATTGCTGCTCCAATCCTCTCAAGA
ATGGTCGGAATGATCAGTGGAACTACCACAGAACAGGAACTGTGG
GATGACTGGGCACCATATGAAGACGTGGAAATTGGACCCAATGGA
GTTCTGAGGACCAGTTCAGGATATAAGTTTCCTTTATACATGATT
GGACATGGTATGTTGGACTCCGATCTTCGTCTTAGCTCAAAGGCT
CAGGTGTTCGAACATCCTCACATTCAAGACGCTGCTTCGCAACTT
CCTGATGATGAGAGTTTATTTTTTGGTGATACTGGGCTATCCAAA
AATCCAATCGAGCTTGTAGAAGGTTGGTTCAGTAGTTGGAAAAGC
TCTATTGCCTCTTTTTTCTTTATCATAGGGTTAATCATTGGACTA
TTCTTGGTTCTCCGAGTTGGTATCCATCTTTGCATTAAATTAAAG
CACACCAAGAAAAGACAGATTTATACAGACATAGAGATGAACCGA
CTTGGAAAGTAA

In certain aspects of the invention, the chimeric glycoprotein consists of the non-coding nucleotide sequence “cgct” preceding the coding SEQ ID NO: 28. SEQ ID NO: 28 is

ATGAAGTGCCTTTTGTACTTAGCCTTTTTATTCATTGGGGTGAAT
TGCagctcaggtggaggcggttcaggcggaggtggctctggcggt
ggcggatctgctagcgatatccagatgacccagtccccgagctcc
ctgtccgcctctgtgggcgatagggtcaccatcacctgccgtgcc
agtcaggatgtgaatactgctgtagcctggtatcaacagaaacca
ggaaaagctccgaaactactgatttactcggcatccttcctctac
tctggagtcccttctcgcttctctggatccagatctgggacggat
ttcaatctgaacatcagcagtctgcagccggaagacttcgcaact
tattactgtcagcaacattatactactcctcccacgttcggacag
ggtaccaaggtggagatcaaaggtggaggtggatcaggtggaggt
ggatcaggtggaggtggatcagaggttcagctggtggagtctggc
ggtggcctggtgcagccagggggctcactccgtttgtcctgtgca
gcttctggcttcaacattaaagacacctatatacactgggtgcgt
caggccccgggtaagggcctggaatgggttgcaaggatttatctt
acgaatggttatactagatatgccgatagcgtcaagggccgtttc
actataagcgcagacacatccaaaaacacaacctacctgcagatg
aacagtctgcgtgctgaggacactgccgtctattattgttctaga
tggggaggggacggcttctatgctatggactactggggtcaagga
accctggtcacagtctcctcgcgtgcggccatcgatgctaaaacg
acggcaccgtctgtctacccactggcacttgtctcgtctggatcc
gggtctctgAAGTTCACCATAGTTTTTCCACACAACAAGAAAGGA
AACTGGAAAAATGTTCCTTCTAATTACCATTATTGCCCGTCAAGC
TCAGATTTAAATTGGCATAATGACTTAATAGGCACAGCCTTACAA
GTCAAAATGCCCCAGAGTCACAAGGCTATTCAAGCAGACGGTTGG
ATGTGTCATGCTTCCAAATGGGTCACTACTTGTGATTTCCGCTGG
TATGGACCGAAGTATATAACACATTCCATCCGATCCTTCACTCCA
TCTGTAGAACAATGCAAGGAAAGCATTGAACAAACGAAACAAGGA
ACTTGGCTGAATCCAGGCTTCCCTCCTCAAAGTTGTGGATATGCA
ACTGTGACGGATGCCGAAGCAGTGATTGTCCAGGTGACTCCTCAC
CATGTGCTGGTTGATGAATACACAGGAGAATGGGTTGATTCACAG
TTCATCAACGGAAAATGCAGCAATGACATATGCCCCACTGTCCAT
AACTCTACAACCTGGCATTCTGACTATAAGGTCAAAGGGCTATGT
GATTCTAACCTCATTTCCACGGACATCACCTTCTTCTCAGAGGAC
GGAAAGCTATCATCCCTGGGAAAGGAGGGCACAGGGTTCAGAAGT
AACTACTTTGCTTATGAAACTGGAGGCAAGGCCTGCAAAATGCAA
TACTGCAAGCATTGGGGAGTCAGACTCCCATCAGGTGTCTGGTTC
AAGATGGCTGATAAGGATCTCTTTGCTGCAGCCAGATTCCCTGAA
TGCCCAGAAGGGTCAAGTATCTCTGCTCCATCTCAGACCTCAGTG
GATGTAAGTCTAATTCAGGACGTTGAGAGGATCTTGGATTATTCC
CTCTGCCAAGAAACCTGGAGCAAAATCAGAGCGGGTCTTCCAATC
TCTCCAGTGGATCTCAGCTATCTTGCTCCTAAAAACCCAGGAACC
GGTCCTGCTTTCACCATAATCAATGGTACCCTAAAATACTTTGAG
ACCAGATACATCAGAGTCGATATTGCTGCTCCAATCCTCTCAAGA
ATGGTCGGAATGATCAGTGGAACTACCACAGAACAGGAACTGTGG
GATGACTGGGCACCATATGAAGACGTGGAAATTGGACCCAATGGA
GTTCTGAGGACCAGTTCAGGATATAAGTTTCCTTTATACATGATT
GGACATGGTATGTTGGACTCCGATCTTCGTCTTAGCTCAAAGGCT
CAGGTGTTCGAACATCCTCACATTCAAGACGCTGCTTCGCAACTT
CCTGATGATGAGAGTTTATTTTTTGGTGATACTGGGCTATCCAAA
AATCCAATCGAGCTTGTAGAAGGTTGGTTCAGTAGTTGGAAAAGC
TCTATTGCCTCTTTTTTCTTTATCATAGGGTTAATCATTGGACTA
TTCTTGGTTCTCCGAGTTGGTATCCATCTTTGCATTAAATTAAAG
CACACCAAGAAAAGACAGATTTATACAGACATAGAGATGAACCGA
CTTGGAAAGTAA.

Additionally, in certain non-limiting aspects of the invention, the presently disclosed viruses can also express various genes, such as, but not limited to, cytokine or chemokines, such as GM-CSF and IL-12, interferon beta, interferon gamma, IL-10, or an agonist or an antagonist thereof, cancer cell antigens, infectious disease antigens, autoimmune disease antigens, or any other viral or mammalian gene.

In certain aspects of the invention, the immunomodulatory agent is selected from the group consisting of anti-PD1 antibodies or antigen-binding fragment thereof, anti-PD-L1 antibodies or an antigen-binding fragment thereof, anti-CTLA-4 antibodies or an antigen-binding fragment thereof, anti-BTLA antibodies or an antigen-binding fragment thereof, anti-TIGIT antibodies or an antigen-binding fragment thereof, anti-TIM3 antibodies or an antigen-binding fragment thereof, anti-LAG-3 antibodies or an antigen-binding fragment thereof, anti-IL-10 or IL-10R antibodies or an antigen-binding fragment thereof, anti-TGF or TGFR antibodies or an antigen-binding fragment thereof, and any combinations thereof. In certain aspects, the immunomodulatory agent is a cytokine such as, but not exclusively confined to, GMCSF, TGF, TNF-α, CD40L, FLT3L, IFN-α, IFNβ, IFN-γ, IL-1, IL-2, IL-4, IL-5, IL-6, IL-7, IL-8, IL-12, IL-15, IL-21, IL-23, MIP-1, and MCP-1. Examples of other genes, include, but is not limited to, urokinase, herpesvirus thymidine kinase (HSV-TK), purine nucleoside phosphorylase, cytosine deaminase, and EGFP.

In certain aspects of the invention, the viruses disclosed herein can also exhibit temperature sensitivity. Additional information on further modifications of the viruses disclosed herein can be found in U.S. Pat. No. 7,429,481, the content of which is incorporated by reference in its entirety.

In certain aspects of the invention, the preparations of the presently disclosed vesicular stomatitis virus (rrVSV) produce high titers. For example, but without any limitation, the presently disclosed rrVSV achieves titers of 1×10⁸/ml. In addition, the preparations can be optimized to higher concentrations and further concentrated.

The present disclosure also contemplates methods of producing the modified virus disclosed herein. In certain aspects of the invention, the methods include generating viral vectors in order to obtain viruses exhibiting improved titer and specificity. In certain aspects of the invention, the methods include subjecting the VSV to one or more serial passages. Non-limiting examples of methods of producing recombinant viruses disclosed herein can be found in Lawson et al. (Lawson et al., Proc. Natl. Acad. Sci. U.S.A., 625(92): 4477-4481 (1995)).

In certain aspects of the invention, in order to provide for expression of a modified surface antigen associated with infectivity and/or ligand and/or therapeutic gene (e.g., cytokine, cytokine agonist or cytokine antagonist), a VSV “stop-start signal” can be operably linked to the gene encoding the surface antigen and/or ligand and/or therapeutic gene. A native start-stop signal occurs in front of each gene in VSV that stops expression of the previous gene and starts expression of the next one using the VSV L polymerase which is packaged with the virus. An Mlu1 site in front of the glycoprotein gene may be used for this purpose, and the following construct may be prepared by PCR: Mlu1-[e.g., therapeutic gene such as GM-CSF]-Stop-Start-Mlu1. This cassette may be inserted into the VSV genome.

### 3.1. Exemplary Vesicular Stomatitis Viruses

In certain non-limiting aspects of the invention, the present disclosure provides a vesicular stomatitis virus including a surface protein associated with infectivity. In certain aspects of the invention, the surface protein associated with infectivity is specific for HER2.

In certain aspects of the invention, the surface protein comprises an antibody or an antigen-binding fragment thereof comprising a variable light chain and a variable heavy chain. In certain aspects of the invention, the variable light chain comprises the amino acid sequence set forth in SEQ ID NO: 1. In certain aspects of the invention, the variable heavy chain comprises the amino acid sequence set forth in SEQ ID NO: 9. In certain aspects of the invention, the surface protein comprises a signal peptide. In certain aspects of the invention, the signal peptide comprises from amino acid 1 to amino acid 16 of SEQ ID NO: 19. In certain aspects of the invention, the surface protein comprises a glycoprotein. In certain aspects of the invention, the glycoprotein comprises the amino acid sequence set forth in SEQ ID NO: 23.

In certain aspects of the invention, the surface protein comprises an antibody or an antigen-binding fragment thereof comprising a variable light chain and a variable heavy chain. In certain aspects of the invention, the variable light chain comprises the amino acid sequence set forth in SEQ ID NO: 7. In certain aspects of the invention, the variable heavy chain comprises the amino acid sequence set forth in SEQ ID NO: 9. In certain aspects of the invention, the surface protein comprises a signal peptide. In certain aspects of the invention, the signal peptide comprises from amino acid 1 to amino acid 16 of SEQ ID NO: 19. In certain aspects of the invention, the surface protein comprises a glycoprotein. In certain aspects of the invention, the glycoprotein comprises the amino acid sequence set forth in SEQ ID NO: 23.

In certain aspects of the invention, the surface protein comprises an antibody or an antigen-binding fragment thereof comprising the amino acid sequence set forth in SEQ ID NO: 11. In certain aspects of the invention, the signal peptide comprises from amino acid 1 to amino acid 16 of SEQ ID NO: 19. In certain aspects of the invention, the surface protein comprises a glycoprotein. In certain aspects of the invention, the glycoprotein comprises the amino acid sequence set forth in SEQ ID NO: 23.

In certain aspects of the invention, the surface protein comprises an antibody or an antigen-binding fragment thereof comprising the amino acid sequence set forth in SEQ ID NO: 17. In certain aspects of the invention, the signal peptide comprises from amino acid 1 to amino acid 16 of SEQ ID NO: 19. In certain aspects of the invention, the surface protein comprises a glycoprotein. In certain aspects of the invention, the glycoprotein comprises the amino acid sequence set forth in SEQ ID NO: 23.

In certain aspects of the invention, the surface protein comprises, from N-end to C-end, a signal peptide, a first linker, an antibody or an antigen-binding fragment thereof, a second linker, and a glycoprotein. In certain aspects of the invention, the surface protein comprises, from N-end to C-end, a signal peptide comprising from amino acid 1 to amino acid 16 of SEQ ID NO: 19, a first linker, an antibody or an antigen-binding fragment thereof comprising the amino acid sequence set forth in SEQ ID Nos: 11, 13, 15, or 17, a second linker, and a glycoprotein comprising the amino acid sequence set forth in SEQ ID NO: 23.

In certain aspects of the invention, the surface protein comprises, from N-end to C-end, a signal peptide, a first linker (e.g., SEQ ID NO: 29), an antibody or an antigen-binding fragment thereof, a second linker (e.g., SEQ ID NO: 31 or 33), and a glycoprotein. In certain aspects of the invention, the surface protein comprises, from N-end to C-end, a signal peptide comprising from amino acid 1 to amino acid 16 of SEQ ID NO: 19, a first linker as set forth in SEQ ID NO: 30, an antibody or an antigen-binding fragment thereof comprising the amino acid sequence as set forth in SEQ ID Nos: 13, 15, or 17, a second linker as set forth in SEQ ID NO: 31 or 33, and a glycoprotein comprising the amino acid sequence as set forth in SEQ ID NO: 23. In certain aspects of the invention, the surface protein comprises, from N-end to C-end, a signal peptide comprising from amino acid 1 to amino acid 16 of SEQ ID NO: 19, a first linker as set forth in SEQ ID NO: 30, an antibody or an antigen-binding fragment thereof comprising the amino acid sequence as set forth in SEQ ID Nos: 13, 15, or 17, a second linker as set forth in SEQ ID NO: 31 or 33, and a glycoprotein comprising the amino acid sequence as set forth in SEQ ID NO: 25.

### 4. Pharmaceutical Compositions

The present disclosure further provides pharmaceutical compositions that include a recombinant virus (e.g., VSV) disclosed herein. Non-limiting examples of the recombinant virus are disclosed herein. In certain aspects of the invention, the pharmaceutical composition includes an effective amount of the recombinant virus. In certain aspects of the invention, the pharmaceutical composition can be prepared as solutions, dispersions in glycerol, liquid polyethylene glycols, and any combinations thereof in oils, in solid dosage forms, as inhalable dosage forms, as intranasal dosage forms, as intradermal injection, as subcutaneous injection, as intra-tumoral injection, as liposomal formulations, dosage forms comprising nanoparticles, dosage forms comprising microparticles, polymeric dosage forms, or any combinations thereof.

In certain aspects of the invention, the pharmaceutical composition described herein further includes a pharmaceutically acceptable carrier, e.g., an excipient. In certain aspects of the invention, the pharmaceutically acceptable carrier includes any carrier which does not interfere with the effectiveness of the biological activity of the active ingredients and/or that is not toxic to the patient to whom it is administered. Non-limiting examples of suitable pharmaceutical carriers include phosphate buffered saline solutions, water, emulsions, such as oil/water emulsions, various types of wetting agents and sterile solutions. Additional non-limiting examples of pharmaceutically acceptable carriers include gels, bioabsorbable matrix materials, implantation elements containing the vesicular stomatitis virus, and any other suitable vehicle, delivery, or dispensing means or material.

In certain aspects of the invention, the pharmaceutically acceptable carrier can be a buffering agent. Non-limiting examples of suitable buffering agents can include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium gluconate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide other calcium salts, and combinations thereof.

In certain aspects of the invention, the recombinant viruses disclosed herein can be propagated in suitable host cells, isolated from host cells, and stored in conditions that promotes stability and integrity of the virus, such that loss of infectivity over time is minimized. In certain aspects of the invention, the recombinant viruses disclosed herein can be stored by freezing or drying, such as by lyophilization. In certain aspects of the invention, prior to administration, the stored recombinant viruses can be reconstituted (if dried for storage) and diluted in a pharmaceutically acceptable carrier for administration.

In certain aspects of the invention, the pharmaceutical composition disclosed herein can further include an immunomodulatory agent (e.g., the immunomodulatory agent disclosed herein). In certain aspects of the invention, the pharmaceutical compositions disclosed herein can be provided systemically or directly to a subject for treating and/or preventing a cancer. In certain aspects of the invention, the presently disclosed recombinant viruses or pharmaceutical compositions are directly injected into an organ of interest (e.g., an organ affected by a cancer or the cancer itself (e.g., intra-tumoral). Alternatively, the presently disclosed recombinant virus or pharmaceutical compositions are provided indirectly to the organ of interest, for example, by administration into the circulatory system (e.g., the systemic vascular system or the tumor vasculature). When administering a therapeutic composition of the presently disclosed subject matter (e.g., a pharmaceutical composition comprising a presently disclosed recombinant virus), it can be formulated in a unit dosage injectable form (solution, suspension, emulsion).

### 5. Methods of Treatment and Prevention

The present disclosure provides methods of treating a subject having a disease, disorder, or condition. The present disclosure also provides methods of preventing a subject from getting or having a disease, disorder, or condition.

The present invention also provides methods of treating or preventing cancer (a subject that has cancer, is at risk of having growth or spread of cancer). In certain aspects of the invention, the methods include administering to the subject a recombinant virus disclosed herein. For example, but not by way of limitation, the methods can include administering to the subject a vesicular stomatitis virus that comprises a nucleic acid that encodes an anti-HER2 antibody of antigen-binding fragment thereof.

In certain aspects of the invention, the method disclosed herein reduces aggregated cancer cell mass, reduces cancer cell growth rate, reduces cancer cell proliferation, reduces tumor mass, reduces tumor volume, reduces tumor weight, reduces tumor cell proliferation, reduces tumor growth rate, and/or reduces tumor metastasis in the subject.

In certain aspects of the invention, the method disclosed herein improves the anti-cancer adaptive immune response in the subject. In certain aspects of the invention, the improved anti-cancer adaptive immune response is dependent on the presence and levels of CD4⁺ and CD8⁺ T cells in the tumor or tumor microenvironment. In certain aspects of the invention, the method disclosed herein improves adaptive T cell-mediated immune responses.

In certain aspects of the invention, the method disclosed herein modulates a tumor microenvironment. Tumor microenvironment is the environment surrounding a tumor. A tumor microenvironment includes extracellular matrix (ECM), fibroblasts, neuroendocrine (NE) cells, adipose cells, immune, signaling molecules, and the blood and lymphatic vascular networks. Cellular interactions, cross talks between cancer and immune cells, and interplays between cells and soluble factors (such as cytokines) in the tumor microenvironment determines a subject's immune response to tumor cells. In certain aspects of the invention, the method disclosed herein modifies the tumor microenvironment. In certain aspects of the invention, the method disclosed herein promotes the immunogenicity of the tumor microenvironment. As used herein, the term “immunogenicity” refers to the ability to induce an immune response against cancer, e.g., cancer cells, tumor cells.

In certain aspects of the invention, the method disclosed herein increases the levels of lymphocytes and/or dendritic cells in the tumor or the tumor microenvironment. In certain aspects of the invention, the method disclosed herein does not increase the level of myeloid-derived suppressor cells or M2 type tumor-associated macrophages in the tumor or the tumor microenvironment. In certain aspects of the invention, the method disclosed herein increases the level of T cells in the tumor or the tumor microenvironment. In certain aspects, the T cells are selected from the group consisting of tumor antigen-specific CD4⁺ T cells, tumor antigen-specific CD8⁺ T cells, viral antigen-specific CD4⁺ T cells, viral antigen-specific CD8⁺ T cells, and combinations thereof.

Methods disclosed herein can be used for treating any suitable cancers. Non-limiting examples of cancers that can be treated by methods disclosed herein include adenocarcinoma, osteosarcoma, cervical carcinoma, melanoma, hepatocellular carcinoma, breast cancers, metastatic breast cancer, lung cancer, prostate cancer, ovarian cancer, leukemia, lymphoma, renal carcinoma, pancreatic cancer, gastric cancer, colon cancer, duodenal cancer, glioblastoma multiforme, astrocytoma, sarcoma, and combinations thereof. In certain aspects of the invention, the method disclosed herein can be used for treating breast cancer. In certain aspects of the invention, the breast cancer is a metastatic breast cancer. In certain aspects of the invention, the breast cancer is a metastatic but dormant breast cancer. In certain aspects of the invention, the method disclosed herein can be used for treating prostate cancer.

In certain aspects of the invention, the methods disclosed herein can be used for treating a HER2-positive cancer. For example, but without any limitation, the recombinant virus of an aspect of the invention (e.g., a VSV), can be used to treat growth or spread of cancer of a HER2-positive metastatic breast cancer.

In certain aspects of the invention, the subject is a human subject. In certain aspects of the invention, the subject is a non-human subject, such as, but not limited to, a non-primate, a dog, a cat, a horse, a rabbit, a mice, a rat, a guinea pig, a fowl, a cow, a goat, or a sheep.

In certain aspects of the invention, the method disclosed herein includes administering the vesicular stomatitis viruses to the subject in an amount of between about between about 10² and 10¹⁰ plaque forming units (PFU). In certain aspects of the invention, the method disclosed herein comprises administering to the subject the recombinant virus of an aspect of the invention (e.g., a VSV), or a pharmaceutical composition comprising a recombinant virus of an aspect of the invention, in a single dose, or in multiple doses. In certain aspects of the invention, where the recombinant viruses or pharmaceutical compositions are administered to the subject in multiple doses, the doses can be administered sequentially, e.g., at daily, weekly, or monthly intervals, or in response to a specific need of the subject.

In certain aspects of the invention, the method disclosed herein comprises administering to the subject a pharmaceutical composition comprising the recombinant viruses of an aspect of the invention (e.g., pharmaceutical compositions disclosed herein).

Any suitable methods of administration can be used with the presently disclosed subject matter for administering the recombinant viruses of an aspect of the invention to the subject having cancer. In certain aspects of the invention, the recombinant viruses disclosed herein is administered systemically. Alternatively or additionally, the recombinant viruses disclosed herein are administered by injection at the site of the cancer, e.g., tumor site. For example, and not by way of limitation, the route of administration can be inhalation, intranasal, intravenous (IV), intraarterial, intrathecal, intratumoral, intraperitoneal (IP), intramuscular, subcutaneous, topical, intradermal, local regional, oral administration, or a combination thereof. In certain aspects, the recombinant viruses disclosed herein are administered to the subject from a source implanted in the subject. In certain aspects of the invention, the recombinant viruses disclosed herein are administered to the subject by continuous infusion over a selected period of time. In certain aspects of the invention, the recombinant viruses disclosed herein are administered directly to a tumor site, e.g., via direct intratumoral injection. In certain aspects of the invention, the recombinant viruses disclosed herein are administered to the subject by IV.

### 5.1 Combinatorial Therapy of Immunotherapy and Viruses

The present disclosure further provides methods for improving a subject's responsiveness to an immunomodulatory agent, comprising administering to the subject the immunomodulatory agent and a recombinant virus disclosed herein, wherein the subject has cancer or is at risk for developing cancer or growth or spread of cancer. For example, but not by way of limitation, a method for improving a subject's responsiveness to an immunomodulatory agent can include administering a recombinant virus disclosed herein, wherein the subject has cancer or is at risk for developing cancer or growth or spread of cancer.

The present disclosure also provides methods of treating a subject having a cancer, including administering to the subject a recombinant virus disclosed herein and an immunomodulatory agent. For example, but not by way of limitation, a recombinant virus disclosed herein can be administered in combination with an immunomodulatory agent to treat a subject that has cancer.

Any suitable immunomodulatory agent that targets components of the immune system to fight cancer can be used with the presently disclosed methods. Non-limiting examples of immunomodulatory agents include immune checkpoint inhibitors, T cells, dendritic cells, therapeutic antibodies (e.g., anti-CD33 antibodies, anti-CD11b antibodies), cancer vaccines, cancer cell antigens, infectious disease antigens, autoimmune disease antigens, cytokines (e.g., IL-12, GM-CSF, IL-2, IFNβ, IFNγ, MIP-1, MCP-1, IL-8), Bacillus Calmette-Guerin (BCG), and any combinations thereof. In certain aspects of the invention, the immunomodulatory agent is an immune checkpoint inhibitor. In certain aspects, of the invention the immune checkpoint inhibitor is selected from anti-PD1 antibodies, anti-PD-L1 antibodies, anti-CTLA-4 antibodies, anti-BTLA antibodies, anti-TIGIT antibodies or antigen-binding fragment thereof, anti-TIM3 antibodies, anti-LAG-3 antibodies, and any combinations thereof. Non-limiting examples of anti-PD1 antibodies include pembrolizumab (KEYTRUDA™), nivolumab (OPDIVO™) cemiplimab (LIBTAYO™), and combinations thereof. Non-limiting examples of anti-PD-L1 antibodies include atezolizumab (TECENTRIQ™), avelumab (BAVENCIO™), durvalumab (IMFINZI™), and combinations thereof. Non-limiting examples of anti-CTLA4 antibodies include ipilimumab (YERVOY™).

An aspect of the invention provides administering to a subject a recombinant virus disclosed herein with cyclophosphamide (Baxter Healthcare Corporation, Deerfield, IL). An aspect of the invention provides administering to a subject a recombinant virus disclosed herein with an anti-CTLA4 Mab. An aspect of the invention provides administering to a subject a recombinant virus disclosed herein with cyclophosphamide and an anti-CTLA4 Mab.

In certain aspects of the invention, the recombinant virus disclosed herein and the immunomodulatory agent can be administered to the subject as part of a treatment regimen. In certain aspects, the recombinant virus disclosed and the immunomodulatory agent can be administered concurrently to the subject. In certain aspects, the recombinant virus disclosed herein and the immunomodulatory agent can be administered at the same time or the immunomodulatory agent can be expressed by the virus. In certain aspects of the invention, the recombinant virus disclosed herein and the immunomodulatory agent can be administered sequentially in any order (e.g., the recombinant virus is administered to the subject before the immunomodulatory agent is administered; or the recombinant virus is administered to the subject after the immunomodulatory agent is administered) or at different points in time (e.g., the recombinant virus and the immunomodulatory agent are administered to the subject on the same day but different hours; the recombinant virus and the immunomodulatory agent are administered to the subject in the same week but on different days).

### 6. Kits

The present invention further provides kits that include a recombinant virus of an aspect of the invention or a pharmaceutical composition including the same.

In certain aspects of the invention, the kit disclosed herein can further include instructions. In certain aspects of the invention, the instructions include a description of the recombinant virus of an aspect of the invention and optionally a description of other components included in the kit. In certain aspects of the invention, the instructions further include a description of methods for administration, including methods for determining the proper state of the subject, the proper dosage amount, and/or the proper administration method for administering the modified virus. In certain aspects of the invention, the instructions further include guidance for monitoring the subject over duration of the treatment time.

In certain aspects of the invention, the kit disclosed herein includes a device for administering the recombinant virus of an aspect of the invention to a subject. Any suitable devices known in the art for administering medications and pharmaceutical compositions can be included in the kits disclosed herein. For example, and not by way of limitation, suitable devices include, a hypodermic needle, an intravenous needle, a catheter, a needle-less injection device, an inhaler and a liquid dispenser, such as an eyedropper. In certain aspects of the invention, a recombinant virus to be delivered systemically, for example, by intravenous injection, can be included in a kit with a hypodermic needle and syringe.

In certain aspects of the invention, the kit disclosed herein can further include an immunomodulatory agent (e.g., immunomodulatory agents disclosed herein). In certain aspects of the invention, the immunomodulatory agent is an immune checkpoint inhibitor. In certain aspects of the invention, the immune checkpoint inhibitor is selected from anti-PD1 antibodies, anti-PD-L1 antibodies, anti-CTLA-4 antibodies, anti-BTLA antibodies, anti-TIGIT antibodies or antigen-binding fragment thereof, anti-TIM3 antibodies, anti-LAG-3 antibodies, and any combination thereof.

Aspects, including embodiments, of the subject matter described herein may be beneficial alone or in combination, with one or more other aspects or embodiments. Without limiting the foregoing description, certain non-limiting aspects of the disclosure numbered 1-49 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) A recombinant virus comprising a recombinant target surface molecule.

(2) The recombinant virus of aspect 1, wherein the recombinant virus is an envelope virus.

(3) The recombinant virus of aspect 1 or 2, wherein the recombinant virus is an RNA envelope virus.

(4) The recombinant virus of any one of aspects 1-3, wherein the recombinant virus is a negative stranded RNA envelope virus.

(5) The recombinant virus of any one of aspects 1-4, wherein the recombinant virus is Vesicular Stomatitis Virus (VSV).

(6) The recombinant virus of any one of aspects 1-5, wherein the recombinant target surface molecule targets a molecule such as a receptor on a tumor cell.

(7) The recombinant virus of any one of aspects 1-6, wherein the recombinant target surface molecule targets HER2 or PSMA.

(8) The recombinant virus of any one of aspects 1-7, wherein the recombinant target surface molecule comprises an antibody or antigen-binding fragment thereof.

(9) The recombinant virus of any one of aspects 1-8, wherein the antibody or antigen-binding fragment thereof comprises a variable light chain comprising an amino acid sequence that is at least about 80% homologous or identical to the amino acid sequence set forth in SEQ ID NO: 1, SEQ ID NO: 3, SEQ ID NO: 5, or SEQ ID NO: 7.

(10) The recombinant virus of any one of aspects 1-9, wherein the antibody or antigen-binding fragment thereof comprises a variable light chain comprising the amino acid sequence set forth in SEQ ID NO: 1, SEQ ID NO: 3, SEQ ID NO: 5, or SEQ ID NO: 7.

(11) The recombinant virus of any one of aspects 1-10, wherein the antibody or antigen-binding fragment thereof comprises a variable light chain comprising the amino acid sequence set forth in SEQ ID NO: 3.

(12) The recombinant virus of any one of aspects 1-11, wherein the antibody or antigen-binding fragment thereof comprises a variable light chain comprising the amino acid sequence set forth in SEQ ID NO: 5.

(13) The recombinant virus of any one of aspects 1-12, wherein the antibody or antigen-binding fragment thereof comprises a variable light chain comprising the amino acid sequence set forth in SEQ ID NO: 7.

(14) The recombinant virus of any one of aspects 1-13, wherein the antibody or antigen-binding fragment thereof comprises a variable heavy chain comprising an amino acid sequence that is at least about 80% homologous or identical to the amino acid sequence set forth in SEQ ID NO: 9.

(15) The recombinant virus of any one of aspects 1-14, wherein the antibody or antigen-binding fragment thereof comprises the amino acid sequence set forth in SEQ ID NO: 9.

(16) The recombinant virus of any one of aspects 1-15, wherein the antibody or antigen-binding fragment thereof comprises an amino acid sequence that is at least about 80% homologous or identical to the amino acid sequence set forth in SEQ ID NO: 11, SEQ ID NO: 13, SEQ ID NO: 15, or SEQ ID NO: 17.

(17) The recombinant virus of any one of aspects 1-16, wherein the antibody or antigen-binding fragment thereof comprises the amino acid sequence set forth in SEQ ID NO: 11, SEQ ID NO: 13, SEQ ID NO: 15, or SEQ ID NO: 17.

(18) The recombinant virus of any one of aspects 1-17, wherein the antibody or antigen-binding fragment thereof comprises the amino acid sequence set forth in SEQ ID NO: 13.

(19) The recombinant virus of any one of aspects 1-17, wherein the antibody or antigen-binding fragment thereof comprises the amino acid sequence set forth in SEQ ID NO: 15.

(20) The recombinant virus of any one of aspects 1-17, wherein the antibody or antigen-binding fragment thereof comprises the amino acid sequence set forth in SEQ ID NO: 17.

(21) The recombinant virus of any one of aspects 1-20, wherein the recombinant target surface molecule comprises a glycoprotein.

(22) The recombinant virus of aspect 21, wherein the glycoprotein comprises an amino acid sequence that is at least about 80% homologous or identical to the amino acid sequence set forth in SEQ ID NO: 21.

(23) The recombinant virus of aspect 22, wherein the glycoprotein comprises the amino acid sequence set forth in SEQ ID NO: 21.

(24) The recombinant virus of aspect 22 or 23, wherein the glycoprotein comprises insertion, deletion, or substitution of one or more amino acid residues.

(25) The recombinant virus of aspect 24, wherein the amino acid residue is at position 10, 47, 156, 184, 194, 238, 354, and 397 of SEQ ID NO: 21.

(26) The recombinant virus of aspect 24 or 25, wherein the glycoprotein comprises:
a) a substitution of a glutamine residue with a lysine residue at position 10 of SEQ ID NO: 21;
b) a substitution of a lysine residue with a glutamine residue at position 47 of SEQ ID NO: 21;
c) a substitution of a tyrosine residue with an aspartic acid residue at position 156 of SEQ ID NO: 21;
d) a substitution of a methionine residue with a threonine residue at position 184 of SEQ ID NO: 21;
e) a substitution of a glutamic acid residue with a lysine residue at position 194 of SEQ ID NO: 21;
f) a substitution of a glutamic acid residue with a lysine residue at position 238 of SEQ ID NO: 21;
g) a substitution of an arginine residue with a glutamine residue at position 354 of SEQ ID NO: 21;
h) a substitution of a histidine residue with an arginine residue at position 397 of SEQ ID NO: 21; or
i) a combination thereof.

(27) The recombinant virus of any one of aspects 24-26, wherein the glycoprotein comprises a substitution of a glutamine residue with a lysine residue at position 10 of SEQ ID NO: 21 and a substitution of a lysine residue with a glutamine residue at position 47 of SEQ ID NO: 21.

(28) The recombinant virus of any one of aspects 24-27, wherein the glycoprotein comprises an amino acid sequence that is at least about 80% homologous or identical to the amino acid sequence set forth in SEQ ID NO: 23.

(29) The recombinant virus of any one of aspects 24-28, wherein the glycoprotein comprises the amino acid sequence set forth in SEQ ID NO: 23.

(30) The recombinant virus of any one of aspects 1-29, wherein the recombinant virus further comprises a start-stop signal operably linked to the recombinant target surface molecule.

(31) The recombinant virus of aspect 30, wherein the start-stop signal is a Mlu1 site.

(32) The recombinant virus of any one of aspects 1-31, wherein the recombinant surface protein comprises a signal sequence.

(33) The recombinant virus of aspect 32, wherein the signal sequence comprises from amino acid 1 to amino acid 16 of SEQ ID NO: 19.

(34) The recombinant virus of any one of aspects 1-33, wherein the recombinant surface protein comprises, from N-end to C-end, a signal peptide, a first linker, an antibody or antigen-binding fragment thereof, a second linker, and a glycoprotein.

(35) The recombinant virus of any one of aspects 1-24, wherein the recombinant surface protein is integrated into the genome of the recombinant virus.

(36) A method of treating a subject having a disorder or condition, the method comprising administering to the subject the recombinant virus of any one of aspects 1-35.

(37) A method of treating a subject having cancer, the method comprising administering to the subject the recombinant virus of any one of aspects 1-35.

(38) The method of aspect 37, wherein the method improves the anti-cancer adaptive immune response in the subject and/or promotes the immunogenicity of a tumor microenvironment of the subject.

(39) The method of aspect 37 or 38, wherein the cancer is selected from the group consisting of adenocarcinoma, osteosarcoma, cervical carcinoma, melanoma, hepatocellular carcinoma, breast cancer, metastatic breast cancer, lung cancer, prostate cancer, ovarian cancer, leukemia, lymphoma, renal carcinoma, pancreatic cancer, gastric cancer, colon cancer, duodenal cancer, glioblastoma multiforme, astrocytoma, sarcoma, and combinations thereof.

(40) The method of any one of aspects 37-39, wherein the cancer is breast cancer, metastatic breast cancer, or dormant breast cancer.

(41) The method of any one of aspects 36-40, further comprising administering to the subject an immunomodulatory agent selected from the group consisting of immune checkpoint inhibitors, T cells, dendritic cells, therapeutic antibodies, cancer vaccines, cancer cell antigens, infectious disease antigens, autoimmune disease antigens, cytokines, Bacillus Calmette-Guerin (BCG), and any combinations thereof.

(42) The method of aspect 41, wherein the immunomodulatory agent is selected from the group consisting of anti-PD1 antibodies or an antigen-binding fragment thereof, anti-PD-L1 antibodies or an antigen-binding fragment thereof, anti-CTLA-4 antibodies or an antigen-binding fragment thereof, anti-BTLA antibodies or an antigen-binding fragment thereof, anti-TIGIT antibodies or an antigen-binding fragment thereof, anti-TIM3 antibodies or an antigen-binding fragment thereof, anti-LAG-3 antibodies or an antigen-binding fragment thereof, and any combinations thereof.

(43) A method for improving a subject's responsiveness to an immunomodulatory agent, the method comprising administering to the subject the immunomodulatory agent and the recombinant virus, of any one of aspects 1-35, wherein the subject has cancer or is at risk of growth or spread of cancer.

(44) A method of preventing a disease, disorder, or condition in a subject, the method comprising administering to the subject the recombinant virus of any one of aspects 1-35.

(45) A method of preventing cancer in a subject, the method comprising administering to the subject the recombinant virus of any one of aspects 1-35.

(46) The method of aspect 45, wherein the recombinant virus of any one of aspects 1-35 is a cancer vaccine.

(47) The method of any one of aspects 36-46, wherein the subject is a human subject.

(48) A pharmaceutical composition comprising the recombinant virus of any one of aspects 1-35.

(49) The pharmaceutical composition of aspect 48, further comprising an immunomodulatory agent.

### EXAMPLES

The presently disclosed subject matter will be better understood by reference to the following Examples, which are provided as exemplary of the presently disclosed subject matter, and not by way of limitation.

### Example 1

This example provides effective vectors of an aspect of the invention, and methods of making such vectors.

The genetic loci in VSV-G that mediate LDL-R binding were eliminated and a sequence coding for SCA-erbb2 added to the beginning of the G-gene using a first linker at the beginning of the SCA and a second linker at end of the SCA. This new replicating recombinant virus, called rrVSV-G, targeted Her2/neu+ cells and tumors and was able to cure larger implanted tumors than the previous SV gp-expressing rrVSV. rrVSV is, along with another rrVSV and a modified HSV-1 (T-VEC), the only virus with attachment glycoproteins engineered to bind to targeted sites and infect targeted cells at high titer while expressing reduced infectivity on normal cells. The viruses of an aspect of the invention can be used in combination with other therapeutic viruses that express different surface glycoproteins, to avoid the problem of pre-existing antibodies, in innovative strategies of treatment and vaccination.

### Material and Methods

Cell lines, antibodies, chemicals, and animals. The following cell lines were used: i) D2F2/E2 cells, a mouse mammary tumor line that has been stably transfected with a vector expressing Her2/neu, and its parent tumor cell line (D2F2); ii) SKBR3, human breast adenocarcinoma cells which over-express Her2/neu; and iii) 143 human osteosarcoma cells which do not express Her2/neu. Cells were grown using standard tissue culture techniques in a humidified incubator at 37° C. with 5% CO₂. BHK-21/1/WI-2 cells were purchased from Kerafast, Inc (Boston, MA 02210) and grown in 6.5% CO₂. Mycoplasma testing for these cell lines was negative using the Impact III PCR profile from IDEXX (RADIL, Columbia, MO, USA). Monoclonal antibody to cytotoxic T-lymphocyte-associated protein 4 (CTLA4) (9H10) was purchased from BioXcell Fermentation/Purification Services (West Lebanon, NIH, USA); cyclophosphamide from Baxter Healthcare Corporation (Deerfield, IL); Bafilomycin A1 from Kamiya Biomedical Co. (Seattle, WA); HERCEPTIN™ from Genentech (South San Francisco, CA); and Balb/c mice from Taconic Biosciences (Hudson, NY).

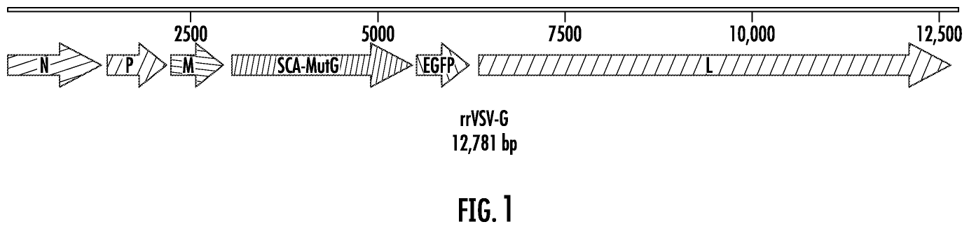

Creation of rrVSV-G and adaptation. A recombinant VSV (rVSV) genome was constructed in which the amino acid loci in VSV-G necessary for full LDL-R binding were eliminated by the following mutations: K47Q and R354Q (Nikolic, et al., Nature Communications, 9: 1029 (2018)). The gene sequence coding for SCA-erbb2 and the first linker and the second linker were placed at the N-terminus, immediately following the 16 amino acid signal sequence (Gao, et al., J. Virol., 80: 8603-12 (2006)). This genome was placed in the pBlueScript vector (pBS-VSV-G-SCA) as illustrated in FIG. 1. A replicating virus was created from vector components as previously described (Bergman, et al., Virology, 330: 24-33 (2004)). In brief, pBS-VSV-G-SCA, pBS-P, pBS-L, pBS-N, and pBS-G were transfected into BHK-21 cells and infected with vTF-7, a vaccinia virus expressing T7 polymerase (NIH AIDS Research and Reference Reagent Program, Rockville, MD). Viruses were harvested from the supernatant 2 days later, passed through a 0.22 μm filter to exclude vaccinia virus, and amplified on BHK-21 cells that were transfected with CMV-VSV-G, using a standard Lipofectamine protocol. The supernatant was harvested two days following infection and stored at −70° C. for adaptation.

Virus adaptation, titer and antibody inhibition. Adaptation was achieved by serially passing virus on Her2/neu over-expressing cells. Virus was passed at a variety of MOI to maximum the production of viruses and avoid defective interfering viruses (Dis). Titers were determined by incubating the virus with the indicated cell lines in one well of a six-well tray (Catalog #3516; Corning, Inc., Corning, NY) for 2 h, washing and replacing the media with Bafilomycin A to prevent replication and counting green cells after 24 h in an inverted fluorescent microscope (Axiovert 135; Carl Zeiss, Inc., Thornwood, NY). Inhibition of viral titer was determined by incubating cells at 4-8° C. first with antibody for 30 min and then with virus and antibody for 60 min. This temperature was chosen to prevent antibody capping and endocytosis into the cell. The cells were then incubated overnight at 37° C., and the titer was measured at 24 h. The percent inhibition was 1−(titer with antibody)/(titer without antibody)×100.

In vitro Growth Assay. D2F2 and D2F2/E2 were plated in a six-well tray at 4×10⁵ cells/well. One day later, individual wells were infected with rrVSV at either a low MOI=0.001 or a high MOI=10 in 1 ml of growth media with 2.5% fetal calf serum (FCS). After a 2 h incubation, the medium was replaced with 2 ml of growth medium with 10% FCS. For the low MOI assay, 100 μl of media were harvested from each well at various time points and frozen at −70° C. for later titer determinations. Fresh media was added to replace each withdrawal. For high MOI assay, the entire well was harvested at each time point. Titers of the yield were determined on D2F2/E2 cells.

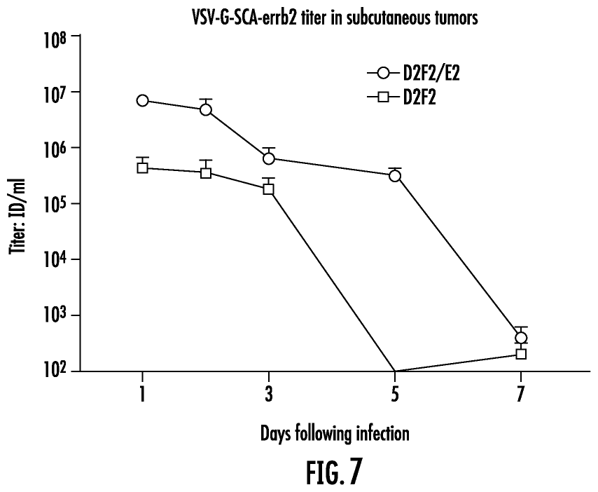

In vivo Growth Assay. Subcutaneous D2F2 and D2F2/E2 were established in mice by implanting 5×10⁵ cells subcutaneously. On day 14, tumors were injected with 1×10⁷ ID rrVSV-G-SCA. Groups of 5 mice were sacrificed on the following days following injection: 1, 2, 3, 5 and 7. Whole tumor nodules were minced, passed through a cell strainer and titered for virus on Her2/neu expressing D2F2/E2 cells (see FIG. 7).

Stability assay. One ml of frozen rrVSV or wtVSV was thawed and incubated in a humidified incubator at 37° C. with 5% CO₂. At various times, 50 μl aliquots were removed and frozen at −70° C. for later titer determination on D2F2/E2 cells.

Sequencing the viral genome. RNA was isolated from 70 μl of virus-containing supernatant using the RNA extraction kit (Catalog #52904; QIAGEN, Inc., Valencia, CA). Viral cDNA for sequencing was produced using the Superscript first-strand synthesis system (Invitrogen, Carlsbad, CA). PCR with VSV-specific primers and Pfu polymerase was used to generate 1-2 kb viral genome fragments for Sanger sequencing by the Genomics Research Core at the University of Pittsburgh. Nucleotide sequences were aligned and analyzed using Sequencher (Gene Codes Corp., Ann Arbor, MI) and Snapgene software (GSL Biotech LLC, San Diego, CA).

Treatment. As previously described, female BALB/c Thy 1.2 mice, 8 to 20 weeks of age, were implanted intraperitoneally (i.p.) with 2×10⁶ D2F2/E2 cells in 300 μl phosphate-buffered saline (PBS). Viral immunotherapy consisted of i.p. rrVSV, 1×10⁸ ID on various days following implant, 200 g anti-CTLA4 i.p. one day later and ˜100 mg/kg cyclophosphamide i.p two days following virus treatment. Cure was determined by survival for more than 100 days after tumor implant. Immune memory to tumor was tested by re-challenging the animals with i.p. D2F2/E2 at the original dose. In one group of animals the experimental endpoint was survival and in another, it was number of anti-tumor tetramer+ CD8 T-cells generated in the peritoneum.

Histopathology. Following sacrifice, the mesentery, omental and retroperitoneal tissues were harvested in a single block, fixed in 10% formalin and embedded in paraffin for histopathologic analysis. Photomicrographs were imaged with a Nikon Eclipse Ci microscope and DS-QiMc camera.

Flow cytometry. 1×10⁶ peritoneal cells were suspended in ice-cold PBS/0.1% bovine serum albumin/0.2% azide and stained with a combination of the following antibodies at a 1:200 dilution: CD4-allophycocyanin-eFluor 780 (eBioscience San Diego, CA, USA), CD8a-phycoerythrin (PE)-cyanine7 (eBioscience), Live-Dead Fixable Red Kit (ThermoFisher Scientific, Pittsburgh, PA, USA) and CD8 human Her2/neu p63 tetramer: H-2K(d)/TYLPTNASL-ALEXA 647 (National Institute of Health Tetramer Core Facility at Emory University, Atlanta, GA). Immunofluorescence was quantified using a LSR Fortesa (Becton Dickinson, Franklin Lakes, NJ)

Statistical analysis. The log-rank statistic was used to compare survival among the treatment groups. An unpaired two-tailed t-test and analysis of variance (ANOVA) were used for all other statistical comparisons. PRISM software was used to analyze the data (GraphPad Software, Inc., La Jolla, CA, USA).

### Results

Replicating recombinant VSV expressing SCA-erbb2(rrVSV-G) preferentially infects Her2 neu over-expressing cancer cells. To develop VSV for clinical use in cancer therapy by restricting its tropism to cells that over-express the Her2/neu receptor, a recombinant VSV (rVSV) genome was constructed in which the amino acid loci in VSV-G necessary for full LDL-R binding were eliminated by the following mutations: K47Q and R354Q (Nikolic, et al., Nature Communications, 9: 1029 (2018)). The gene sequence coding for SCA-erbb2, the first linker, and the second linker was placed at the N-terminus, immediately following the 16 amino acid signal sequence. Previous studies have shown that the 9 amino acid RGD and the 49 amino acid echistatin could be added at this location in the genome to generate a functional virus (Ammayappan, et al., J. Virol., 87: 13543-55 (2013)). A separate gene encoding green fluorescent protein (EGFP) was placed following the G-gene in the genome. The genome of this virus is illustrated in FIG. 1.

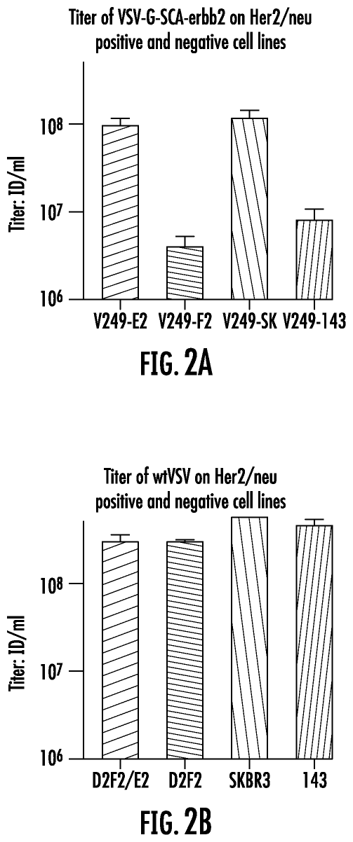

A replication-competent VSV virus was created from this genome whose only coat protein was the disabled VSV-G gp with a domain expressing SCA-erbb2. This virus was called rrVSV-G-SCA-erbb2 (rrVSV-G) and showed a 15- to 25-fold higher titer on human and mouse cell lines that expressed Her2/neu compared to matched cell lines that did not (FIGS. 2A and 2B for the final adapted virus). On the other hand, infection with wtVSV-EGFP showed no specificity for Her2/neu expressing cells and yielded similar titers in both cell lines from each pair (FIGS. 2A and 2B). The titer of rrVSV-G was reduced 70-fold from wtVSV on cells that did not express Her2/neu (Nikolic, et al., Nature Communications, 9: 1029 (2018)).

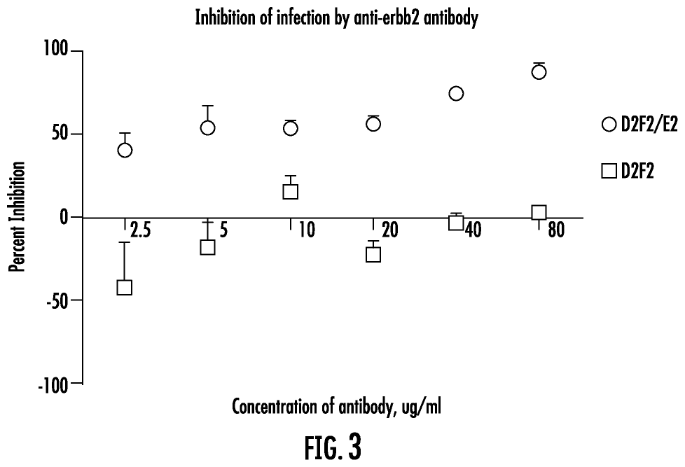

Incubation of the virus in the presence of HERCEPTIN™, an antibody directed to the erbb2 receptor on the cell surface, reduced titer on D2F2/E2, the cell line expressing erbb2 but had no effect on titer on D2F2, the parent cell line that did not express erbb2 (FIG. 3). Inhibition was antibody dose-dependent and reached a maximum of 87% inhibition at an antibody concentration of 80 ug/ml.

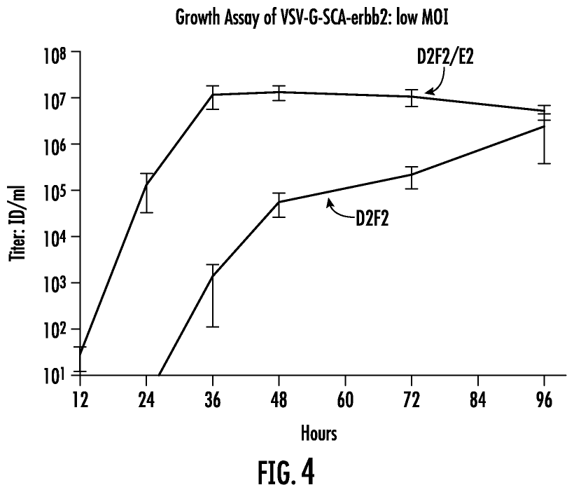

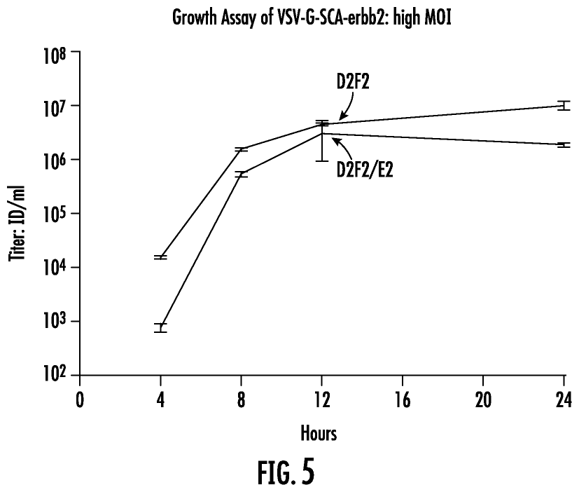

The yield from a low-MOI infection of VSV-G-SCA-erbb2 on cell lines differing only on expression of Her2/neu showed much faster growth on the Her2/neu expressing cells (FIG. 4). The maximum yield was obtained at 36 hours on the Her2/neu expressing cells and 96 hours on the Her2/neu negative cells, though the final maximum yield was similar. These results were obtained by titering aliquots and the finding of faster early growth on the Her2/neu expressing cells was confirmed by counting green cells at various time after infection (data not shown). In contrast, the yield from a high-MOI infection of VSV-G-SCA-erbb2 was similar on Her2/neu expressing and non-expressing cells (FIG. 5), suggesting that the faster early growth on Her2/neu expressing cells was due to faster entry into the cells rather than faster replication within the cells or easier release from the cells. The most likely explanation is that the SCA-erbb2 promoted binding to the Her2/neu receptor and facilitated early entry into the cell.

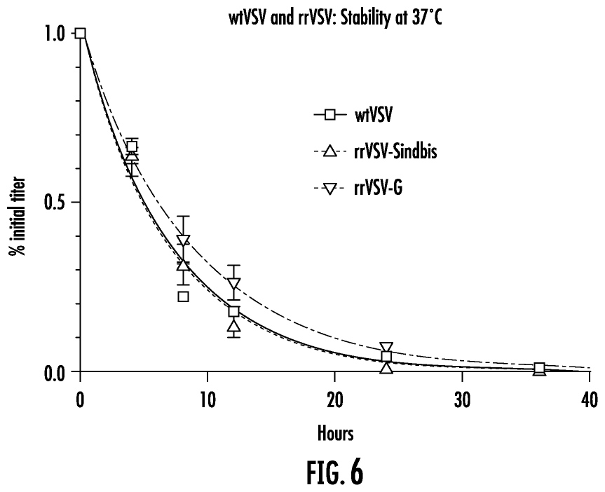

Stability, in vitro, at 37° C. of the new targeted virus, rrVSV-G, was excellent and did not differ significantly from wtVSV or previously described targeted VSV, VSV-Sindbis-SCA-erbb2, which has excellent anti-tumor therapeutic efficacy and generates potent anti-tumor memory T-cells (FIG. 6) (Gao, et al., Neuro. Oncol., 17: 536-44 (2015); Gao, et al., Anticancer Res., 35: 4593-7 (2015); Gao, et al., Cancer Gene Ther., 16: 44-52 (2009); Gao, et al., Cancer Gene Ther., 19: 282-91 (2012)).

To determine whether VSV-G preferentially infected Her2/neu expressing cells in vivo as well as in vitro, implanted subcutaneous tumors in mice were injected with 1×10⁷ ID and groups of 5 mice were sacrificed on the following days following injection: 1, 2, 3, 5 and 7. Whole tumor nodules were minced, passed through a cell strainer and titered for virus on Her2/neu expressing D2F2/E2 cells. The area under the curve (AUC) was significantly higher when VSV-G infected the Her2/neu expressing D2F2/E2 tumor than the Her2/neu negative D2F2 tumor (unpaired t-test: p=0.03). The maximum titer was 15-fold higher on D2F2/E2 than D2F2 and virus persisted at a titer >1×10⁵ ID for 5 days in D2F2/E2 compared with 3 days in D2F2.

In vivo therapeutic trials using rrVSV. It has been previously shown that rrVSV with a modified Sindbis gp expressing GM-CSF, rrVSV-Sindbis-SCA-erbb2 (rrVSV-Sindbis) combined with single doses of anti-CTLA4 Mab and cyclophosphamide cures 3 day implanted peritoneal tumors (Gao, et al., Cancer Gene Ther., 16: 44-52 (2009)). Cure is immunologically dependent and requires both CD4 and CD8 T-cells. Cure generates memory T-cells by 90 days after treatment that can be transferred to cure new peritoneal tumors.

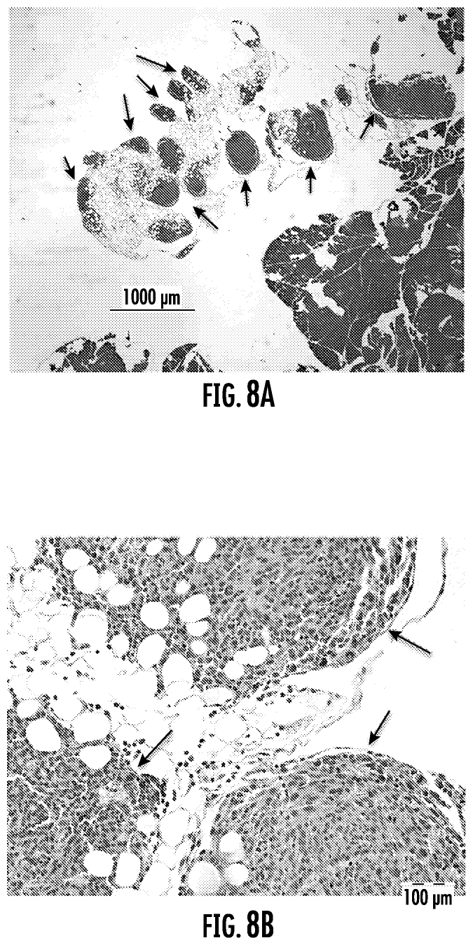

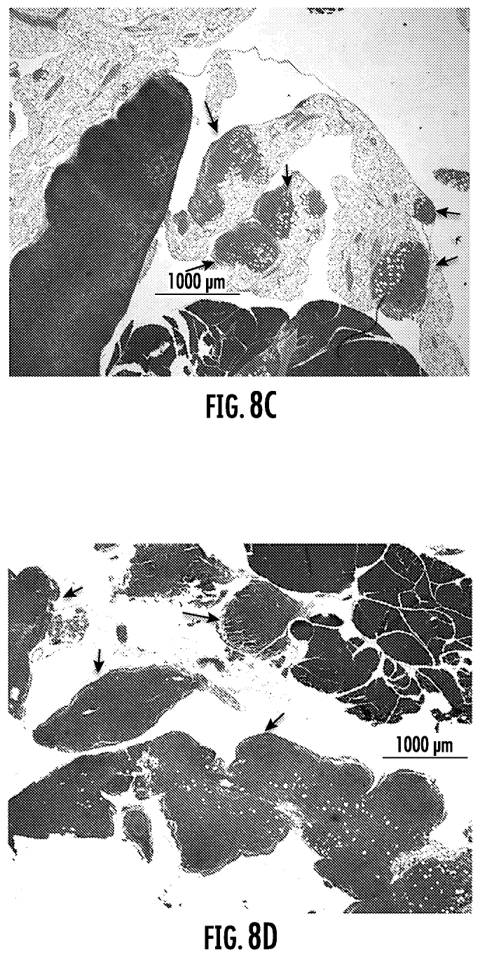

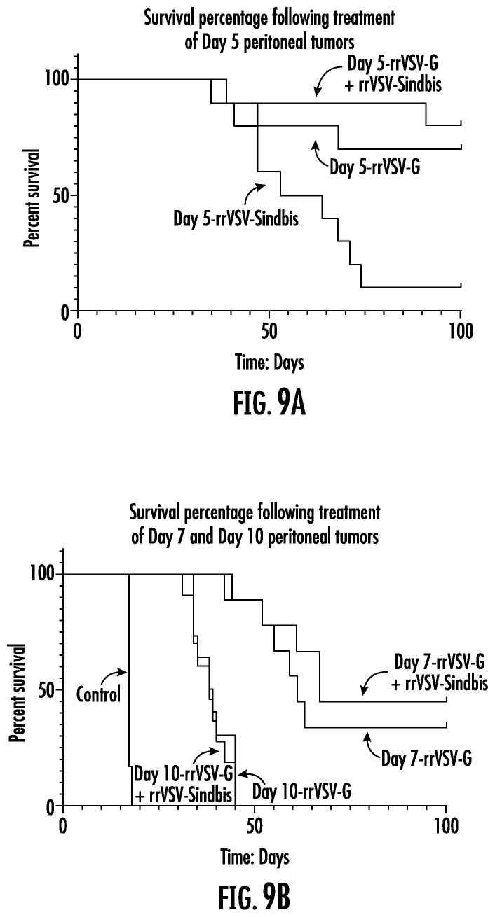

To compare the efficacy of the Sindbis gp based previous rrVSV with the new G gp based rrVSV that did not express GM-CSF, D2F2/E2, a BALB/c mouse breast cancer cell line stably transfected to express the Her2/neu receptor, was implanted into the peritoneum of 6-14-week-old BALB/c mice. Histopathologic examination of the mesentery showed multiple tumor nodules of steadily increasing size following implantation (FIGS. 8A-8D). Both rrVSV-Sindbis and G-rrVSV cured 100% of day 3 tumors, and all no-treatment control animals died with large tumors by day 18. The rrVSV-Sindbis cured only 1 of 10 animals treated with day 5 tumor compared with 7 of 10 treated with the new G-rrVSV (log rank statistic: p=0.01; FIG. 9A). The combination of both viruses cured 8 of 10. Remarkably, G-rrVSV, cured 3 of 9 very large 7-day tumors and doubled survival from 17 to 38.5 days in mice with 10-day tumors (log rank statistic: p<0.0001, for both 7-day and 10-day tumors) (FIG. 9B). There was no evidence that combination therapy with both viruses improved outcome, but the new G-rrVSV was therapeutically more effective than the previous rrVSV-Sindbis despite not expressing GM-CSF. These data suggest that the immuno-onco-therapeutic effects of the G-rrVSV are greater than the rrVSV-Sindbis.

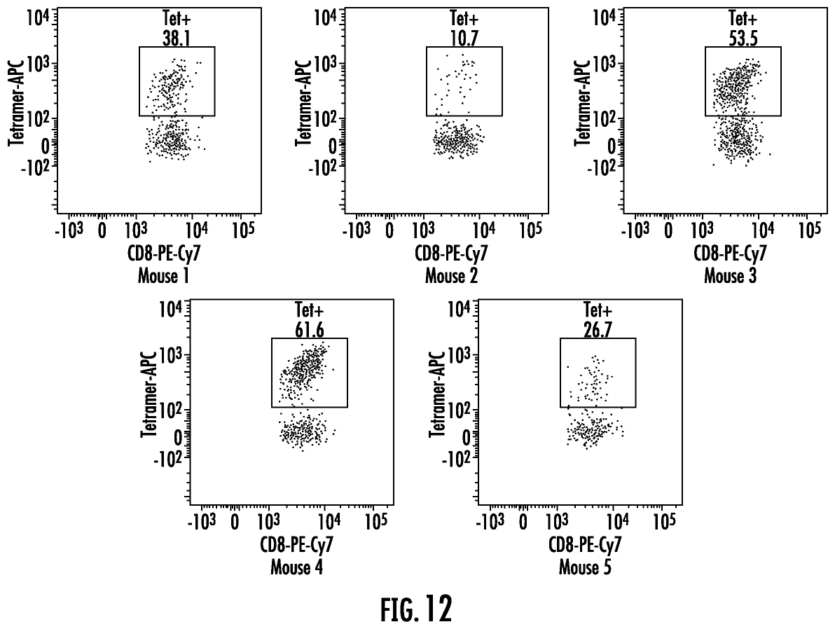

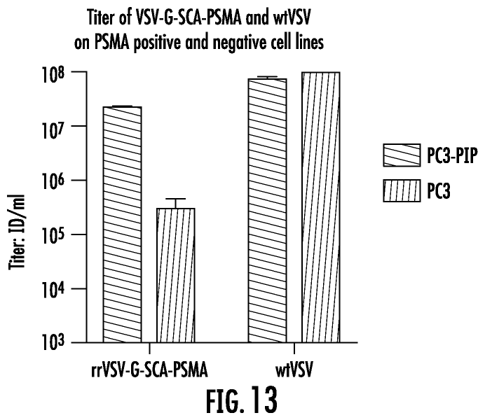

Eight animals, cured with rrVSV-G alone, were rechallenged with (IP) D2F2/E2 cells, and none developed tumor, indicating the development of anti-tumor immunity. The specific antitumor memory CD8⁺ T-cell response of another 8 cured animals (5 with rrVSV-G alone and 3 with both rrVSV-G and rrVSV-Sindbis) was quantified by flow cytometry analyses using a tetramer displaying the immunodominant p63 epitope of the Her2/neu protein (see, e.g., FIG. 12). Cured mice were challenged once with IP D2F2/E2 cells and sacrificed 5 days later with harvest of peritoneal lavage cells. The number of tetramer-positive CD8 T-cells averaged 9.97×10⁴, similar to the 8.36×10⁴ found previously following cure with the rrVSV-Sindbis alone (Gao et al., Anticancer Res., 38: 6621-6629 (2018)). Tetramer⁺ CD8 T-cells accounted, on average, for 24% of total peritoneal CD8 T-cells.

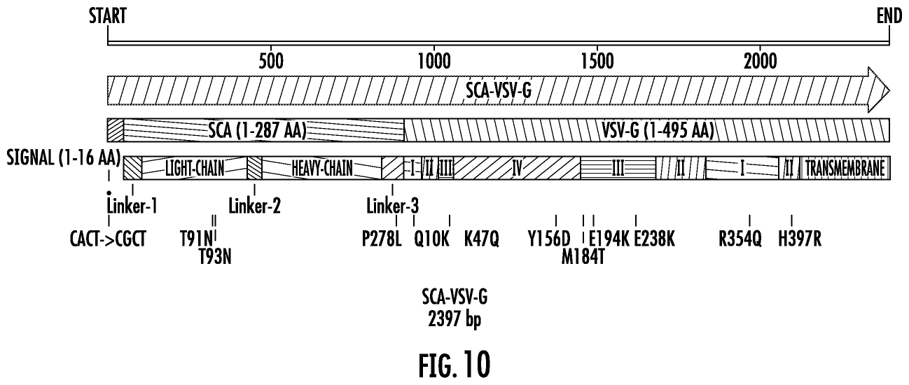

Serial passage to adapt newly created rrVSV-G. rrVSV-G was created on BHK-21 cells from vector components as previously described (Bergman, et al., Virology, 330: 24-33 (2004)). As experienced by others when making recombinant VSV (Ammayappan, et al., J. Virol., 87: 13543-55 (2013)), growth was initially poor on both SKBR3, a human breast cancer cell line expressing Her2/neu and D2F2/E2, a mouse mammary tumor cell line stably transfected to express Her2/neu. For the first 25 passages, cells were passed on D2F2/E2, SKBR3 or a mouse cell line from a mammary tumor in a Tg FVB mouse expressing human Her2/neu from an MMTV promoter (Finkle, et al., Clinical Cancer Research, 10: 2499-2511 (2004)). Thereafter, virus was passed exclusively on D2F2/E2 cells. At passage 105, adaptation was continued by the same methodology on 2 separate tracks which did not communicate. Adaptation was complete in one track at passage 190 and in the other at passage 139. Throughout the adaptation, output was improved by culturing the infected cells at 32° C. and gradually increasing the temperature with each passage to a goal temperature of 37 TC. Virus was determined by titer and the G-SCA compound protein sequenced at various passages (FIG. 10 and Table 2).

TABLE 2

| | Virus | | | | | | | | | | |
|---|---|---|---|---|---|---|---|---|---|---|---|
| | Titer | | Mutation-first identified | | | | | | | | |
| Generations | DEF2/E2 | D2F2 | Non-Coding | SCARegion | | G-glycoprotein region | | | | | |
| 5 | 1.44E+03 | 4.07E+03 | | | | | | | | | |
| 28 | 3.75E+06 | 2.57E+04 | | | P278L | | | M184T | | | H397R |
| 114 | 2.08E+07 | 1.64E+06 | CACT−>CGCT | | | Q10K | Y156D | | E194K | E238K | |
| 190A | 9.67E+07 | 3.85E+06 | | T91N | | | | | | | |
| 139B | 9.40E+07 | 6.31E+06 | | T93N | | | | | | | |

Notably, the rrVSV-G specificity for Her2/neu expressing cells was present with the first viral titer and did not change consistently as viral titer increased during the selection process. Interestingly, a single mutation in 2 related sites increased the titer of the virus from 2.1×10⁷ ID/ml to 9.5 and 9.7×10⁷ IID/ml. The mutation was in the SCA domain and eliminated an O-glycosylation site and created an N-glycosylation consensus sequence. The same mutation was created in both tracks of the final adaptation in different amino acids (AA) separated by only a single amino acid (T91 vs T93). Both were in the V_{L} domain of the SCA but neither was in the hypervariable region of V_{L} as detailed in GenBank: AAS07025.1 and AIL24996.1. This was the only mutation within the SCA protein itself. There was a mutation in the linker connecting the C-terminus of the SCA to the G-protein and another mutation immediately preceded the signal sequence of the G-protein. These latter mutations can easily be incorporated into new SCAs combined with G to speed the adaptation process and the Threonine sites that were mutated in the non-hypervariable V_{L} region appears to have homologies in other SCA (Ward, et al., Appl. Environ. Microbiol., 70: 2567-76 (2004)).

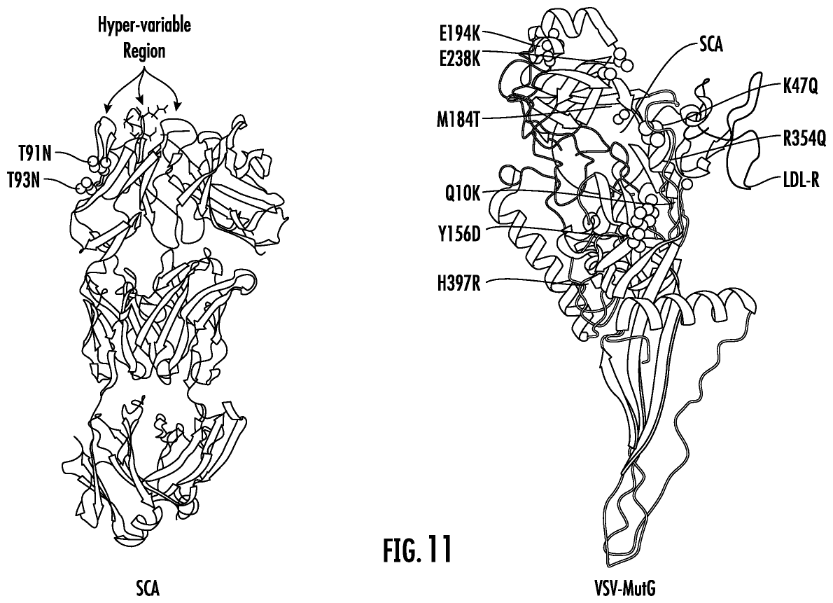

There were 6 mutations in the G-protein. Two sites (E238K and M184T) have previously been identified as important in adaption of VSV to new cell lines and can be considered for mutation when making new SCA-G constructs (Seegers, et al., J. Virol., 94: 17 (2020)). The location of each of the 6 mutations is marked on the crystal structure in FIG. 11. None are known to affect the fusion properties or the conformational change of VSV-G. (Roche et al., Cell Mol. Life Sci., 65: 1716-28 (2008); Beilstein et al., Cell Reports, 32: 108042 (2020)).

### Discussion

Viruses can selectively kill tumor cells and stimulate an immune reaction to the tumor cells. Many viruses are in pre-clinical development for cancer therapy, some are in clinical trials, and one is approved for clinical use against inoperable malignant melanoma, Talimogene Laherparepvec (HSV-1 T-VEC) (Bommareddy, et al., Am. J. Clin. Dermatol., 18: 1-15 (2017)). A highly effective, widely applicable therapeutic approach has not yet been found, and new tools and strategies deserve development. In an aspect of the invention, a new immune-stimulating oncolytic virus has been created. A targeted rrVSV was created from a genomic construct that eliminated the LDL-R binding site of VSV G and added an SCA recognizing the Her2/neu receptor (SCA-erbb2). The initial poorly growing virus was adapted by serial passage on Her2/neu expressing cancer cells resulting in a high-titer virus with preferred binding on Her2/neu expressing cancer cells. This rrVSV was able to eliminate very large, implanted multinodular peritoneal tumors and generate permanent anti-tumor immunity.

rrVSV-G-SCA-erbb2 joins a small set of oncolytic viruses with tumor specificity based on selective binding of the virus surface glycoprotein to a cancer cell receptor (Bergman, et al., Virology, 330: 24-33 (2004); Ammayappan, et al., J. Virol., 87: 13543-55 (2013); Belousova, et al., J. Virol., 82:630-7 (2008); Menotti, et al., Proc. Natl Acad. Sci. U.S.A., 106: 9039-44 (2009); Breton, et al., Molecular Therapy Oncolytics, 2: 15012 (2015); Betancourt, et al., J. Virol., 89: 11786-800 (2015); Hanauer, et al., Oncotarget., 9: 12971-12981 (2018); Bah, et al., Mol. Cancer Ther., 19: 2057-2067 (2020); Petrovic, et al., J. Virol., 92: 15 (2018)). Only 3 of these viruses disabled native binding while adding specific binding. On non-targeted cells, the titer of the recombinant virus was 10⁴ to 10⁵-fold lower than wt virus for two recombinants (Menotti, et al., Proc. Natl Acad. Sci. U.S.A., 106: 9039-44 (2009); Betancourt, et al., J. Virol., 89: 11786-800 (2015)) and 50-fold lower for one (Bergman, et al., Virology, 330: 24-33 (2004)). On targeted cells, the titer of the VSV-HIV gp chimera was ˜5×10⁴ ID/ml (Betancourt, et al., J. Virol., 89: 11786-800 (2015)); the HSV-SCA was ˜5×10⁷ ID/ml (Menotti, et al., Proc. Natl Acad. Sci U.S.A., 106: 9039-44 (2009)); VSV-Sindbis-SCA was 1×10⁸ ID/ml (Gao, et al., J. Virol., 80: 8603-12 (2006)) and the current VSV-G-SCA was 9.67×10⁷ ID/ml. The advantage of the current rrVSV is that it grows to high titer, has shown better immuno-therapeutic effects than rrVSV-Sindbis-SCA and uses a base virus, VSV, that is safe and has not infected most of the population, who therefore do not have pre-existing antibodies to the virus. The VSV-HIV gp chimera grew to a titer of only ˜5×10⁴ ID/ml and the HSV-SCA has limited clinical potential because HSV-1 has already infected most of the population generating potent anti-viral antibodies and the recombinant HSV, though disabled, remains a potentially dangerous neurotropic and latent virus. There are several additional potential benefits of the new rrVSV-G. It can be combined with other oncolytic virus for heterologous treatment or vaccination that boosts the anti-tumor immune response. The safety of rrVSV could be enhanced by combining the current targeted glycoprotein with several safety features already in use for VSV including a mutated M protein that does not inhibit the cellular interferon response to virus infection and an inserted gene expressing interferon (Velazquez-Salinas, et al., Hum. Gene Ther., 28: 108-115 (2017); Stojdl, et al., Cancer Cell, 4: 263-275 (2003)). The current SCA-Her2/neu can be easily replaced with SCAs targeting different cells to use viral lysis and immune stimulation to treat a wide variety of neoplastic, autoimmune and other diseases. The targeted G-SCA construct can be used with any virus that has a targeting glycoprotein to target specific antigens on the surface of many types of cells (e.g., tumor cells). Many cytokines can be added to these targeted VSV or other viruses and the virus can be either replicating or non-replicating.

Newly created rrVSV often grow only to low titer and serial passage is required to evolve a virus with clinically useful titer (Gao, et al., J. Virol., 80: 8603-12 (2006); Ammayappan, et al., J. Virol., 87: 13543-55 (2013); Seegers, et al., J. Virol., 94:17 (2020). Previously 5, 15, and 32 passages were sufficient but the current virus required 139 passages in one track and 190 in another. Mutation analysis showed 3 mutations in the SCA and adjoining sites and 8 mutations in the G protein that could be included in future recombinant constructions that would likely speed the adaptation process to high titer recombinant VSV. The current rrVSV-G disabled viral growth on non-Her2/neu expressing cells by 98.6% compared to wtVSV, a biologic effect that amplified the 10% loss of binding found in vitro by eliminating the amino acid loci in VSV-G necessary for full LDL-R binding: K47A and Q354A (Nikolic, et al., Nature Communications, 9: 1029 (2018)). As other sites in VSV-G necessary for cell binding and infection are identified and mutated, new rrVSV constructs with more complete specificity can be expected.

### Example 2

This example provides another vector of an aspect of the invention, and shows that the vector is effective at treating prostate cancer.

Currently, treatments are available to prolong life in patients with prostate cancer, but cure rates are dismal (Batra, et al., Translational andrology and urology, 8(3): S263-S264 (2019)). Accordingly, there is a need for new prostate cancer treatments. Virus rrVSV-G-SCA-PSMA was created to meet this need. The genome of rrVSV-G-SCA-PSMA is similar to the virus shown in FIG. 1, except that SCA PSMA has been substituted for SCA-erbb2.

Specifically, rrVSV-G-SCA-PSMA comprises replication-competent VSV whose only coat protein is a disabled VSV-G glycoprotein (gp) with an additional domain expressing a single chain antibody (SCA) targeting prostate-specific membrane antigen (PSMA).

PSMA has been proven to be an effective therapeutic target for prostate cancer in preclinical studies and is being tested in a Phase 1 human clinical trial using PSMA-targeted CAR T-cells (an entirely different type of immunotherapy than the inventive recombinant virus) (Narayan, et al., Nature Medicine, 28(4): 724-734 (2022); Zuccolotto, et al., PLoS ONE, 9(10): e109427 (2014)).

rrVSV-G-SCA-PSMA specifically targets PSMA expressing cancer cells as shown in FIG. 13. rrVSV-G-SCA-PSMA showed a 66-fold higher titer on a human prostate cancer cell line that expressed PSMA compared to the same cell line that did not (FIG. 13). On the other hand, infection with wild type VSV (wtVSV) showed no specificity for PSMA expressing cells and yielded similar titers in both cell lines (FIG. 13). The titer of rrVSV-G-SCA-PSMA was reduced 415-fold compared with wtVSV on cells that did not express PSMA, indicating that native binding of VSV was almost completely abolished on the new recombinant VSV with a disabled G glycoprotein. This data shows that rrVSV-G-SCA-PSMA specifically targets cells expressing PSMA.

Specificity of rrVSV-G-SCA-PSMA was also demonstrated by a growth assay showing excellent growth of rrVSV-G-SCA-PSMA on cells expressing PSMA and exceptionally poor growth on the same cells not expressing PSMA (FIG. 14). The maximal yield at 72 hours was 1874-fold higher on the PSMA expressing cells. Almost all PC3-PIP cells (PC3 cells (ATCC CRL-1435) transduced with VSV-G pseudotyped lentiviral vector expressing human PSMA) were dead at 72 hours whereas the great majority of PC3 cells remained alive at this time.

The titer of rrVSV-G-SCA-PSMA on its first infection in PSMA-expressing cells, immediately after being created, was 2.06×10⁷/ml compared with the first titer of rrVSV-G-SCA-erbb2, prior to adaptation, which was 1.44×10⁵/ml, a difference of 143-fold. The prior work with a recombinant virus of an aspect of the invention, rrVSV-G-SCA-erbb2, allowed for rapid creation of rrVSV-G-SCA-PSMA, which immediately showed a high titer combined with high specificity to a specific prostate cancer cell expressing PSMA.

Together, Examples 1 and 2 show that the VSV of an aspect of the invention, as described herein, improved specific viral titer more than 600-fold as compared to the unmodified VSV.

The recombinant viruses of an aspect of the invention have been shown to be effective against two different cancer types using two different cancer cell surface receptors. These are examples only, and the recombinant viruses of an aspect at the invention can be used to quickly and easily create targeted viruses to attack any harmful cell (including cancer cells) in the subject with a unique or highly expressed cell surface molecule.

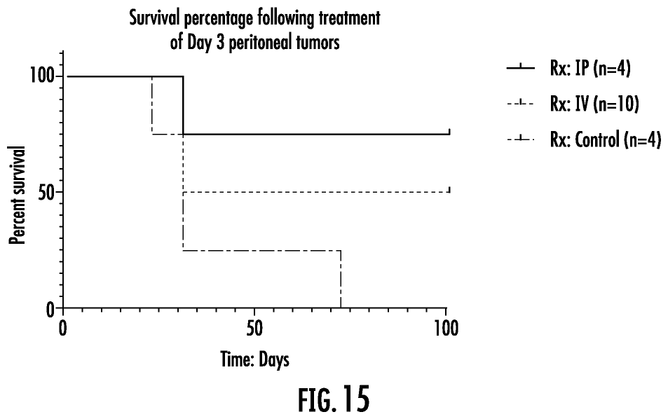

Further, these examples show that the recombinant viruses of an aspect of the invention can be effectively co-administered with other treatments (such as anti-CTLA4 Mab and cyclophosphamide). In addition, these examples show that the recombinant viruses of an aspect of the invention can be effectively administered intraperitoneally (IP) (Example 1) and intravenously (IV) (Example 2, FIG. 15).

Although the presently disclosed subject matter and certain of its advantages have been described in detail, it should be understood that various changes, substitutions and alterations can be made herein without departing from the spirit and scope of the disclosure. Moreover, the scope of the present application is not intended to be limited to the particular aspects of the process, machine, manufacture, and composition of matter, and methods described in the specification. As one of ordinary skill in the art will readily appreciate from the disclosure of the presently disclosed subject matter, processes, machines, manufacture, compositions of matter, or methods, presently existing or later to be developed that perform substantially the same function or achieve substantially the same result as the corresponding aspects described herein may be utilized according to the presently disclosed subject matter. Accordingly, the appended claims are intended to include within their scope such processes, machines, manufacture, compositions of matter, or methods.

Various patents, patent applications, publications, product descriptions, protocols, and sequence accession numbers are cited throughout this application, the disclosures of which are incorporated herein by reference in their entireties for all purposes.

1. A recombinant virus comprising a recombinant target surface molecule.

2. The recombinant virus of claim 1, wherein the recombinant virus is an envelope virus.

3. The recombinant virus of claim 1, wherein the recombinant virus is an RNA envelope virus.

4. The recombinant virus of claim 1, wherein the recombinant virus is a negative stranded RNA envelope virus.

5. The recombinant virus of claim 1, wherein the recombinant virus is Vesicular Stomatitis Virus (VSV).

6. The recombinant virus of claim 1, wherein the recombinant target surface molecule targets a molecule on a tumor cell.

7. The recombinant virus of claim 1, wherein the recombinant target surface molecule targets HER2 or PSMA.

8. The recombinant virus of claim 1, wherein the recombinant target surface molecule comprises an antibody or antigen-binding fragment thereof.

9. The recombinant virus of claim 8, wherein the antibody or antigen-binding fragment thereof comprises a variable light chain comprising an amino acid sequence that is at least about 80% homologous or identical to the amino acid sequence set forth in SEQ ID NO: 1, SEQ ID NO: 3, SEQ ID NO: 5, or SEQ ID NO: 7.

10.-13. (canceled)

14. The recombinant virus of claim 8, wherein the antibody or antigen-binding fragment thereof comprises a variable heavy chain comprising an amino acid sequence that is at least about 80% homologous or identical to the amino acid sequence set forth in SEQ ID NO: 9.

15. (canceled)

16. The recombinant virus of claim 8, wherein the antibody or antigen-binding fragment thereof comprises an amino acid sequence that is at least about 80% homologous or identical to the amino acid sequence set forth in SEQ ID NO: 11, SEQ ID NO: 13, SEQ ID NO: 15, or SEQ ID NO: 17.

17-20. (canceled)

21. The recombinant virus of claim 1, wherein the recombinant target surface molecule comprises a glycoprotein.

22. The recombinant virus of claim 21, wherein the glycoprotein comprises an amino acid sequence that is at least about 80% homologous or identical to the amino acid sequence set forth in SEQ ID NO: 21.

23. (canceled)

24. The recombinant virus of claim 22, wherein the glycoprotein comprises insertion, deletion, or substitution of one or more amino acid residues.

25. The recombinant virus of claim 24, wherein the amino acid residue is at position 10, 47, 156, 184, 194, 238, 354, and 397 of SEQ ID NO: 21.

26. The recombinant virus of claim 24, wherein the glycoprotein comprises:
a) a substitution of a glutamine residue with a lysine residue at position 10 of SEQ ID NO: 21;
b) a substitution of a lysine residue with a glutamine residue at position 47 of SEQ ID NO: 21;
c) a substitution of a tyrosine residue with an aspartic acid residue at position 156 of SEQ ID NO: 21;
d) a substitution of a methionine residue with a threonine residue at position 184 of SEQ ID NO: 21;
e) a substitution of a glutamic acid residue with a lysine residue at position 194 of SEQ ID NO: 21;
f) a substitution of a glutamic acid residue with a lysine residue at position 238 of SEQ ID NO: 21;
g) a substitution of an arginine residue with a glutamine residue at position 354 of SEQ ID NO: 21;
h) a substitution of a histidine residue with an arginine residue at position 397 of SEQ ID NO: 21; or
i) a combination thereof.

27.-29. (canceled)

30. The recombinant virus of claim 1, wherein the recombinant virus further comprises a start-stop signal operably linked to the recombinant target surface molecule.

31. (canceled)

32. The recombinant virus of claim 1, wherein the recombinant target surface molecule comprises a signal sequence.

33-35. (canceled)

36. A method of treating a subject having a disease, disorder, or condition, or preventing a disease, disorder, or condition in a subject, the method comprising administering to the subject the recombinant virus of claim 1.

37. A method of treating a subject having cancer, or preventing cancer in a subject, the method comprising administering to the subject the recombinant virus of claim 1.

38.-42. (canceled)

43. A method for improving a subject's responsiveness to an immunomodulatory agent, the method comprising administering to the subject the immunomodulatory agent and the recombinant virus of claim 1, wherein the subject has cancer or is at risk of growth or spread of cancer.

44.-47. (canceled)

48. A pharmaceutical composition comprising the recombinant virus of claim 1.

49. (canceled)
